(19) United States
(12) Patent Application Publication
(10) Pub. No.: US 2024/0101821 A1
(43) Pub. Date: Mar. 28, 2024

(54) POLYMERIC COMPOSITION

(71) Applicant: Imperial College Innovations Limited, London (GB)

(72) Inventors: Robin Shattock, London (GB); Molly Stevens, London (GB); Yunqing Zhu, London (GB); Anna Blakney, London (GB)

(73) Assignee: Imperial College Innovations Limited, London (GB)

(21) Appl. No.: 17/767,824

(22) PCT Filed: Oct. 9, 2020
(86) PCT No.: PCT/GB2020/052521

§ 371 Date: Apr. 8, 2022

(30) Foreign Application Priority Data
Oct. 10, 2019 (GB) 1914659.6

(51) Int. Cl. C08L 79/02; C07C 323/41; C08G 73/02

(52) U.S. Cl. CPC C08L 79/02; C07C 323/41; C08G 73/0253; C08G 73/028

(57) ABSTRACT

The invention relates to polymeric compositions, nanoparticles and vaccines comprising polymeric compositions. The invention extends to medical uses of the polymeric compositions, nanoparticles and vaccines. The invention further extends to methods of producing the polymeric compositions and nanoparticles.

The present invention relates to polymeric compositions, nanoparticles and vaccines comprising polymeric compositions. The invention extends to medical uses of the polymeric compositions, nanoparticles and vaccines. The invention further extends to methods of producing the polymeric compositions and nanoparticles.

Due to progress in manufacturing and delivery, nucleic acids have emerged as an easily scalable and cost-effective vaccination strategy.^{1,2} Messenger RNA (mRNA) has several advantages as a nucleic acid platform compared to DNA; there is no risk of integration into the host genome, innate sensing can be modulated through base modifications and delivery vehicles, and it is the minimal genetic vector.³⁻⁶ Furthermore, constructs targeting strain diversity or multiple infectious diseases can easily be combined.⁷ Self-amplifying mRNA (saRNA), derived from the alphavirus genome,⁸ is particularly advantageous as a vaccine platform, as it self-replicates upon delivery into the cytoplasm which results in augmented protein expression and a minimum required dose of RNA.⁹⁻¹¹ However, because saRNA is a relatively large (˜9,500 nt), negatively charged molecule, it requires a delivery vehicle for efficient cellular uptake.

saRNA has previously been delivered using cationic emulsions,¹² lipid nanoparticles,¹⁰ and polymers.^{9,13} However, these delivery platforms were initially developed and optimized for shorter nucleic acids, such as siRNA (˜20 nt) and mRNA (˜2,000-5,000 nt) and thus may not be the optimal formulation for saRNA. A recent report by Blakney et al. observed that the chain length and charge density of a commonly used cationic polymer, poly(ethylene imine) (pEI), strongly impacted in vitro transfection efficiency, and that optimal polymers for mRNA and pDNA were not necessarily optimal for saRNA.¹⁴

In accordance with a first aspect of the invention, there is provided a polymeric composition comprising a plurality of polymers of formula (I):

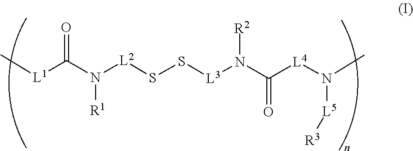

wherein L¹ to L⁵ are each independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms; L⁶ and L⁷ are independently an optionally substituted C₁₋₁₂ alkylene, an optionally substituted C₂₋₁₂ alkenylene, an optionally substituted C₂₋₁₂ alkylnyene, an optionally substituted C₃₋₆ cycloalkylene, an optionally substituted 3 to 8 membered heterocyclylene, an optionally substituted C₆₋₁₂ arylene or an optionally substituted 5 to 10 membered heteroarylene, wherein adjacent carbon atoms in the alkylene, alkenylene or alkynylene are optionally interrupted by one or more heteroatoms;
R¹ and R² are each independently H, an optionally substituted C₁₋₁₂ alkyl, an optionally substituted C₂₋₁₂ alkenyl or an optionally substituted C₂₋₁₂ alkynyl;
R³ is —OR⁴, —COOR⁴, —SO₂OR⁴, (OCH₂CH₂)ₘOH, or NR⁴R⁵,
R⁴ and R⁵ are each independently H, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl, wherein adjacent carbon atoms in the alkyl, alkenyl or alkenyl are optionally interrupted by one or more heteroatoms; and
m is an integer between 1 and 10;
or a pharmaceutically acceptable complex, salt, solvate, tautomeric form or polymorphic form thereof;
characterised in that the average molecular mass of the plurality of polymers of formula (I) is greater than 5 kg mol⁻¹.

Advantageously, the inventors have been able to synthesise pABOLs with higher molar masses than was possible using prior art methods.

The term “alkyl”, as used herein, unless otherwise specified, refers to a saturated straight or branched hydrocarbon. In certain embodiments, the alkyl group is a primary, secondary, or tertiary hydrocarbon. In certain embodiments, the alkyl group includes one to six carbon atoms, i.e. C₁-C₆ alkyl. C₁-C₆ alkyl includes for example methyl, ethyl, n-propyl (1-propyl) and isopropyl (2-propyl, 1-methylethyl), butyl, pentyl, hexyl, isobutyl, sec-butyl, tert-butyl, isopentyl, neopentyl, and isohexyl.

“Alkenyl” refers to olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl.

“Alkynyl” refers to acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl.

The term “alkylene”, as used herein, unless otherwise specified, refers to a bivalent saturated straight or branched hydrocarbon. In certain embodiments, the alkylene group is a primary, secondary, or tertiary hydrocarbon. In certain embodiments, the alkylene group includes one to six carbon atoms, i.e. C₁-C₆ alkylene. C₁-C₆ alkylene includes for example methylene, ethylene, n-propylene and isopropylene, butylene, pentylene, hexylene, isobutylene, sec-butylene, tert-butylene, isopentylene, neopentylene, and isohexylene.

The term “alkenylene”, as used herein, unless otherwise specified, refers to a bivalent olefinically unsaturated straight or branched hydrocarbon. In certain embodiments, the alkenylene group is a primary, secondary, or tertiary hydrocarbon. In certain embodiments, the alkenylene group includes one to six carbon atoms, i.e. C₂-C₆ alkenylene. C₂-C₆ alkenylene includes for example ethenylene, propenylene, butenylene, pentenylene or hexenylene.

The term “alkynylene”, as used herein, unless otherwise specified, refers to a bivalent acetylenically unsaturated straight or branched hydrocarbon. In certain embodiments, the alkynylene group is a primary, secondary, or tertiary hydrocarbon. In certain embodiments, the alkynylene group includes one to six carbon atoms, i.e. C₂-C₆ alkynylyne. C₂-C₆ alkynylene includes for example ethynylene, propynylene, butynylene, pentynylene or hexynylene.

“Cycloalkyl” refers to a non-aromatic hydrocarbon 3 to 6 membered ring system. The cycloalkyl may be saturated or partially saturated and may be monocyclic, bicyclic or polycyclic. The cycloalkylene may be saturated or partially saturated and may be monocyclic, bicyclic or polycyclic. Representative examples of a C₃-C₆ cycloalkyl include, but are not limited to, cyclopropyl, cyclobutyl, cyclopentyl, cyclohexyl.

“Cycloalkylene” refers to a bivalent, non-aromatic, hydrocarbon 3 to 6 membered ring system. The cycloalkylene may be saturated or partially saturated and may be monocyclic, bicyclic or polycyclic.

“Heterocycle” or “heterocyclyl” refers to a 3 to 8 membered monocyclic, bicyclic or bridged molecules in which at least one ring atom is a heteroatom. The or each heteroatom may be independently selected from the group consisting of oxygen, sulfur and nitrogen. A heterocycle may be saturated or partially saturated. Exemplary 3 to 8 membered heterocyclyl groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane, oxazine.

“Heterocyclylene” refers to a 3 to 8 membered bivalent monocyclic, bicyclic or bridged molecules in which at least one ring atom is a heteroatom. The or each heteroatom may be independently selected from the group consisting of oxygen, sulfur and nitrogen.

“Aryl” refers to an aromatic 6 to 12 membered hydrocarbon group. Examples of a C₆-C₁₂ aryl group include, but are not limited to, phenyl, α-naphthyl, β-naphthyl, biphenyl, tetrahydronaphthyl and indanyl.

“Arylene” refers to a bivalent aromatic 6 to 12 membered hydrocarbon group.

“Heteroaryl” refers to a monocyclic or bicyclic aromatic 5 to 10 membered ring system in which at least one ring atom is a heteroatom. The or each heteroatom may be independently selected from the group consisting of oxygen, sulfur and nitrogen. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring.

“Heteroarylene” refers to a bivalent monocyclic or bicyclic aromatic 5 to 10 membered ring system in which at least one ring atom is a heteroatom. The or each heteroatom may be independently selected from the group consisting of oxygen, sulfur and nitrogen.

The alkyl, alkenyl, alkynyl, alkylene, alkenylene, alkynylene, cycloalkyl, cycloalkylene, heterocyclyl, heterocyclylene, aryl, arylene, hereoaryl and heteroarylene groups can be unsubstituted or substituted with one or more of halogen, OR¹⁴, NR¹⁴R¹⁵, C₁₋₆ alkyl, C₂₋₆ alkenyl, C₂₋₆ alkynyl, C₃₋₆ cycloalkyl, 3 to 8 membered heterocyclyl, C₆₋₁₂ aryl or 5 to 10 membered heteroaryl, where R¹⁴ and R¹⁵ are independently C₁₋₆ alkyl, C₂₋₆ alkenyl, C₂₋₆ alkynyl, C₃₋₆ cycloalkyl, 3 to 8 membered heterocyclyl, C₆₋₁₂ aryl or 5 to 10 membered heteroaryl.

A halogen may be fluorine, chlorine, bromine or iodine.

Preferably, L¹ to L⁴ are each independently an optionally substituted C₁₋₆ alkylene, an optionally substituted C₂₋₆ alkenylene or an optionally substituted C₂₋₆ alkynylene, and more preferably L to L⁴ are each independently an optionally substituted C₁₋₃ alkylene, an optionally substituted C₂₋₃ alkenylene or an optionally substituted C₂₋₃ alkynylene. The alkylene, alkenylene or alkynylene may be substituted with a halogen. Preferably, the alkylene, alkenylene or alkynylene is unsubstituted. In a preferred embodiment, L to L⁴ are each —CH₂CH₂—.

Preferably, L⁵ is an optionally substituted C₂₋₈ alkylene, an optionally substituted C₂₋₈ alkenylene or an optionally substituted C₂₋₈ alkynylene, and more preferably L⁵ is an optionally substituted C₃₋₅ alkylene, an optionally substituted C₃₋₅ alkenylene or an optionally substituted C₃₋₅ alkynylene. The alkylene, alkenylene or alkynylene may be substituted with a halogen. Preferably, the alkylene, alkenylene or alkynylene is unsubstituted. In a preferred embodiment, L⁵ is —CH₂CH₂CH₂CH₂—.

Preferably, R¹ and R² are independently H, an optionally substituted C₁₋₆ alkyl, an optionally substituted C₂₋₆ alkenyl or an optionally substituted C₂₋₆ alkynyl, and more preferably are independently H, an optionally substituted C₁₋₃ alkyl, an optionally substituted C₂₋₃ alkenyl or an optionally substituted C₂₋₃ alkynyl. The alkyl, alkenyl or alkynyl may be substituted with a halogen. Preferably, the alkyl, alkenyl or alkynyl is unsubstituted. In a preferred embodiment, R¹ and R² are H.

R³ is preferably NR⁴R⁵.

Preferably, R⁴ and R⁵ are independently H, an optionally substituted C₁₋₆ alkyl, an optionally substituted C₂₋₆ alkenyl or an optionally substituted C₂₋₆ alkynyl, and more preferably are independently H, an optionally substituted C₁₋₃ alkyl, an optionally substituted C₂₋₃ alkenyl or an optionally substituted C₂₋₃ alkynyl. The alkyl, alkenyl or alkynyl may be substituted with a halogen. Preferably, the alkyl, alkenyl or alkynyl is unsubstituted. In a preferred embodiment, R⁴ and R⁵ are H.

Accordingly, the plurality of polymers of formula (I) may be a plurality of polymers of formula (Ia):

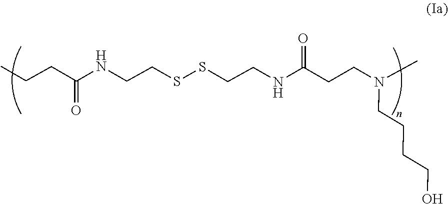

or a pharmaceutically acceptable complex, salt, solvate, tautomeric form or polymorphic form thereof.

The plurality of polymers of formula (I) or (Ia) may be a pharmaceutically acceptable salt.

The term “pharmaceutically acceptable salt” may be understood to refer to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, adepic, aspartic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminium ion, or alkali metal or alkaline earth metal hydroxides, such as sodium, potassium, calcium, magnesium, aluminium, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts may include, sodium, potassium, calcium, magnesium, ammonium, tetraalkylammonium and the like, and when the compound contains a basic functionality, salts of non-toxic organic or inorganic acids, such as hydrohalides, e.g. hydrochloride, hydrobromide and hydroiodide, carbonate or bicarbonate, sulfate or bisulfate, borate, phosphate, hydrogen phosphate, dihydrogen phosphate, pyroglutamate, saccharate, stearate, sulfamate, nitrate, orotate, oxalate, palmitate, pamoate, acetate, trifluoroacetate, trichloroacetate, propionate, hexanoate, cyclopentylpropionate, glycolate, glutarate, pyruvate, lactate, malonate, succinate, tannate, tartrate, tosylate, sorbate, ascorbate, malate, maleate, fumarate, tartarate, camsylate, citrate, cyclamate, benzoate, isethionate, esylate, formate, 3-(4-hydroxybenzoyl)benzoate, picrate, cinnamate, mandelate, phthalate, laurate, methanesulfonate (mesylate), methylsulphate, naphthylate, 2-napsylate, nicotinate, ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluceptate, gluconate, glucoronate, hexafluorophosphate, hibenzate, benzoate, glutamate, hydroxynaphthoate, salicylate, stearate, cyclohexylsulfamate, quinate, muconate, xinofoate and the like.

Hemisalts of acids and bases may also be formed, for example, hemisulphate salts.

In one embodiment, the plurality of polymers of formula (I) may be hydrochloric salts. Accordingly, the plurality of polymers of formula (Ia) may be a plurality of polymers of formula (Iai):

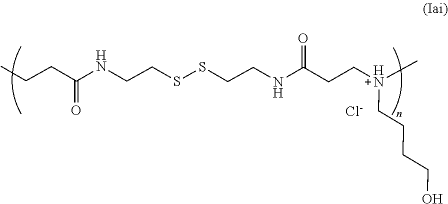

It may be appreciated that the above polymer is poly(CBA-4-amino-1-butanol) (pABOL).

The term “solvate” may be understood to refer to a compound provided herein or a salt thereof, that further includes a stoichiometric or non-stoichiometric amount of solvent bound by non-covalent intermolecular forces. Where the solvent is water, the solvate is a hydrate.

The molecular mass may be characterized using an Agilent PL GPC-50 instrument, equipped with a refractive index (RI) detector, with HPLC grade DMF (containing 0.075 wt % LiBr) as the eluent at a flow rate of 1.0 mL min⁻¹ at 40° C. Two GRAM Linear columns may be used in series. Near monodispersed poly(methyl methacrylate) standards may be used to calibrate the instrument. The composition comprising the plurality of polymers of formula (I) may be dissolved in HPLC grade DMF, containing 0.075 wt % LiBr, and filtered through 0.2 μm syringe filters prior to analysis.

The average molecular mass of the plurality of polymers of formula (I) may be at least 5.5 kg mol⁻¹, at least 6 kg mol⁻¹, at least 7 kg mol⁻¹, at least 8 kg mol⁻¹ or at least 9 kg mol⁻¹.

The average molecular mass of the plurality of polymers of formula (I) may be at least 10 kg mol⁻¹, at least 20 kg mol⁻¹, at least 30 kg mol⁻¹, at least 40 kg mol⁻¹ or at least 50 kg mol⁻¹. The average molecular mass of the plurality of polymers of formula (I) may be at least 75 kg mol⁻¹, at least 100 kg mol⁻¹, at least 125 kg mol⁻¹, at least 150 kg mol⁻¹ or at least 160 kg mol⁻¹.

The average molecular mass of the plurality of polymers of formula (I) may be between 5.5 and 1,000 kg mol⁻¹, between 5.5 and 900 kg mol⁻¹, between 6 and 800 kg mol⁻¹, between 7 and 700 kg mol⁻¹, between 8 and 600 kg mol⁻¹ or between 9 and 500 kg mol⁻¹. The average molecular mass of the plurality of polymers of formula (I) may be between 10 and 450 kg mol⁻¹, between 20 and 400 kg mol⁻¹, between 30 and 350 kg mol⁻¹, between and 300 kg mol⁻¹ or between 50 and 250 kg mol⁻¹. In some embodiments, the average molecular mass of the plurality of polymers of formula (I) may be between 75 and 225 kg mol⁻¹, between 100 and 200 kg mol⁻¹, between 125 and 190 kg mol⁻¹, between 150 and 180 kg mol⁻¹ or between 160 and 170 kg mol⁻¹.

In one embodiment, the average molecular mass of the plurality of polymers of formula (I) between 5.5 and 100 kg mol⁻¹, between 6 and 50 kg mol⁻¹, between 6.5 and 25 kg mol⁻¹, between 7 and 20 kg mol⁻¹, between 7.5 and 15 kg mol⁻¹, between 7.7 and 10 kg mol⁻¹, between 7.8 and 9 kg mol⁻¹, or between 7.9 and 8.5 kg mol⁻¹. As explained in the examples, the inventors have found that pABOL with an average molecular mass of about 8 kg mol⁻¹ is surprisingly effective at delivering self-amplifying RNA (saRNA).

For each of the polymers of formula (I), n may be an integer. However, n may vary within the plurality of polymers of formula (I). Accordingly, an average n value may be calculated for the plurality of polymers. It may be appreciated that the average n value may be calculated based upon the values of a, b, c, d and e and the average molecular mass. For instance, if the plurality of polymers are polymers of formula (Iai) and the average molecular mass is 8 then the average n value would be about 21.

The average n value may be at least 13, at least 14, at least 16, at least 18, at least 20 or at least 22. The average n value may be at least 25, at least 50, at least 75, at least 100 or at least 125. The average n value may be at least 200, at least 250, at least 300, at least 350 or at least 400.

The average n value may be between 13 and 2500, between 14 and 2400, between 16 and 2200, between 18 and 2000, between 20 and 1750 or between 22 and 1500. The average n value may be between 25 and 1250, between 50 and 1100, between 75 and 1000, between 100 and 850 or between 125 and 700. The average n value may be between 200 and 600, between 250 and 550, between 300 and 500, between 350 and 475 or between 400 and 450.

In one embodiment, the average n value may be between 13 and 450, between 14 and 250, between 15 and 125, between 16 and 75, between 17 and 50, between 18 and 40, between 19 and 30, between 20 and 25 or between 20.3 and 22.5.

In accordance with a second aspect, there is provided a composition of matter comprising the polymeric composition according to the first aspect and a nucleic acid.

The weight ratio of polymeric composition and the nucleic acid may be between 1:1 and 200:1, more preferably between 5:1 and 150:1 or between 10:1 and 100:1, and most preferably between 20:1 and 90:1, between 30:1 and 80:1, between 40:1 and 70:1 or between 45:1 and 60:1.

The nucleic acid may be DNA, RNA or a DNA/RNA hybrid sequence. Preferably, the nucleic acid is DNA or RNA.

Most preferably, the nucleic acid is RNA. The RNA may be single stranded or double stranded. The RNA may be selected from the group consisting of: messenger RNA (mRNA), micro RNA (miRNA); short interfering RNA (siRNA); short hairpin RNA (shRNA); self-amplifying RNA (saRNA); interference RNA; and small RNA. Preferably, the RNA is self-amplifying RNA (saRNA).

The skilled person would appreciate that self-amplifying RNAs may contain the basic elements of mRNA (a cap, 5′ UTR, 3′UTR, and poly(A) tail of variable length), but may be considerably longer (for example 9-12 kb).

The nucleic acid sequence, preferably RNA, and most preferably saRNA, may be at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length at least 10000 bases in length, at least 11000 bases in length or at least 12000 bases in length.

Preferably, the nucleic acid sequence is at least 6000 bases in length.

Preferably, the RNA is at least 6000 bases in length.

Most preferably, the saRNA is at least 6000 bases in length.

The nucleic acid sequence, preferably RNA, and most preferably saRNA, may be between 5000 and 20000 bases in length, between 5000 and 15000 bases in length, between 5000 and 14000 bases in length, between 5000 and 13000 bases in length, between 5000 and 12000 bases in length, between 5000 and 11000 bases in length, between 5000 and 10000 bases in length, between 6000 and 20000 bases in length, between 6000 and 15000 bases in length, between 6000 and 14000 bases in length, between 6000 and 13000 bases in length, between 6000 and 12000 bases in length between, between 6000 and 11000 bases in length, between 6000 and 10000 bases in length, between 7000 and 20000 bases in length, between 7000 and 15000 bases in length, between 7000 and 14000 bases in length, between 7000 and 13000 bases in length, between 7000 and 12000 bases in length, between 7000 and 11000 bases in length, between 7000 and 10000 bases in length, between 8000 and 20000 bases in length, between 8000 and 15000 bases in length, between 8000 and 14000 bases in length, between 8000 and 13000 bases in length, between 8000 and 12000 bases in length, between 8000 and 11000 bases in length, between 8000 and 10000 bases in length, between 9000 and 20000 bases in length, between 9000 and 15000 bases in length, between 9000 and 14000 bases in length, between 9000 and 13000 bases in length, between 9000 and 12000 bases in length, between 9000 and 11000 bases in length or between 9000 and 10000 bases in length.

Preferably, the nucleic acid sequence is between 6000 and 15000 bases in length. Preferably the nucleic acid sequence is between 8000 and 12000 bases in length.

Preferably, the RNA is between 6000 and 15000 bases in length. Preferably the RNA is between 8000 and 12000 bases in length.

Preferably, the saRNA is between 6000 and 15000 bases in length. Preferably the saRNA is between 8000 and 12000 bases in length.

The skilled person would appreciate that when the nucleic acid is double stranded, for example double stranded RNA, “bases in length” will refer to the length of base pairs.

Preferably, the RNA comprises or is derived from a positive stranded RNA virus selected from the group of genus consisting of: alphavirus; picornavirus; flavivirus; rubivirus; pestivirus; hepacivirus; calicivirus or coronavirus.

Suitable wild-type alphavirus sequences are well-known. Representative examples of suitable alphaviruses include Aura, Bebaru virus, Cabassou, Chikungunya virus, Eastern equine encephalomyelitis virus, Fort Morgan, Getah virus, Kyzylagach, Mayaro, Mayaro virus, Middleburg, Mucambo virus, Ndumu, Pixuna virus, Ross River virus, Semliki Forest, Sindbis virus, Tonate, Triniti, Una, Venezuelan Equine encephalomyelitis, Western equine encephalomyelitis, Whataroa and Y-62-33.

Preferably, the RNA comprises or is derived from a virus selected from the group of species consisting of: Venezuelan Equine encephalitis Virus (VEEV); enterovirus 71; Encephalomyocarditis virus; Kunjin virus; and Middle East respiratory syndrome virus. Preferably, the vector is derived from VEEV.

Preferably, the nucleic acid comprises a sequence which encodes the at least one therapeutic biomolecule. The at least one therapeutic biomolecule may comprise or be a vaccine construct, or a therapeutic protein. The skilled person would understand that therapeutic protein relates to any protein that has therapeutic application, preferably in human. Exemplary therapeutic biomolecules that can be encoded by the nucleic acid include proteins and peptides derived from pathogens, such as bacteria, viruses, fungi, protozoa/or parasites. Preferably, the protein and peptide is an antigen.

The protein and peptide derived from a virus may be a viral antigen. The viral antigen may be derived from a virus selected from the group consisting of Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainkuenza virus types 1-4 (PIV), Mumps, Sendai viruses, Simian virus 5, Bovine parainkuenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses.

The protein and peptide derived from bacteria may be a bacterial antigen.

The bacterial antigen may derived from a bacterium selected from the group consisting of: Neisseria meningitides, Streptococcus pneumoniae, Streptococcus pyogenes, Moraxella catarrhalis, Bordetella pertussis, Burkholderia sp. {e.g., Burkholderia mallei, Burkholderia pseudomallei and Burkholderia cepacia), Staphylococcus aureus, Haemophilus inkuenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae J. Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcusfaecalis, Enterococcus faecium, Helicobacter pylori, Staphylococcus saprophyticus, Yersinia enter ocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp.

The protein and peptide derived from a fungus may be a fungal antigen.

The fungal antigen may be derived from a fungus selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

The protein and peptide derived from a protozoan may be a protozoan antigen.

The protozoan antigen may be derived from a protozoan selected from the group consisting of: Entamoeba histolytica, Giardia lambli, Cryptosporidium parvum, Cyclospora cayatanensis, Plasmodium species (vivax, ovale, malariae), Leishmania (donovani, tropica), Trypanosoma (brucei and cruzi) and Toxoplasma.

The protein and peptide derived from a helminth may be a helminth antigen.

The helminth antigen may be derived from a protozoan selected from the group consisting of: Ascaris lumbricoides, Trichuris trichiura, Necator americanus, Strongyloides stercoralis and Ancylostoma duodenale, Hymenolepis nana, Taenia saginata, Enterobius, Fasciola hepatica, Schistosoma mansoni, Toxocara canis and Toxocara cati

The therapeutic biomolecule may be a protein and peptide derived from a plant. Preferably, the protein and peptide is a plant antigen. The plant antigen may be derived from Ricinus communis.

In one embodiment the antigen may be an allergen. Allergens in this context include e.g. grasses, pollens, moulds, drugs, or numerous environmental triggers, etc. Allergy antigens typically belong to different classes of compounds, such as nucleic acids and their fragments, proteins or peptides and their fragments, carbohydrates, polysaccharides, sugars, lipids, phospholipids, etc.

In another embodiment, the therapeutic biomolecule may be an immunogen or an antigen. Preferably the immunogen or an antigen is a tumour immunogen or antigen, or cancer immunogen or antigen. The tumour immunogens and antigens may be peptide-containing tumour antigens, such as a polypeptide tumour antigen or glycoprotein tumour antigens.

The tumour antigens may be (a) full length molecules associated with cancer cells, (b) homologs and modified forms of the same, including molecules with deleted, added and/or substituted portions, and (c) fragments of the same.

Suitable tumour immunogens include: class I-restricted antigens recognized by CD 8+ lymphocytes or class II-restricted antigens recognized by CD4+ lymphocytes.

The tumour antigen may be an antigen that is associated with a cancer selected from the group consisting of: a testis cancer, melanoma, lung cancer, head and neck cancer, NSCLC, breast cancer, gastrointestinal cancer, bladder cancer, colorectal cancer, pancreatic cancer, lymphoma, leukaemia, renal cancer, hepatoma, ovarian cancer, gastric cancer and prostate cancer.

The tumour antigen may be selected from:
(a) cancer-testis antigens such as NY-ESO-I, SSX2, SCPl as well as RAGE, BAGE, GAGE and MAGE family polypeptides, for example, GAGE-I, GAGE-2, MAGE-I, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours);
(b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUMl (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT;
(c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), P53 (associated with, e.g., breast, colon cancer), and carcinoembryonic antigen (associated with, e.g., breast cancer, lung cancer, and cancers of the gastrointestinal tract such as colorectal cancer);
(d) shared antigens, for example, melanoma-melanocyte differentiation antigens such as MART-1/Melan A, gplOO, MC1R, melanocyte-stimulating hormone receptor, tyrosinase, tyrosinase related protein-1/TRPl and tyrosinase related protein-2/TRP2 (associated with, e.g., melanoma);
(e) prostate-associated antigens, such as PAP, PSA, PSMA, PSH-Pl, PSM-Pl, PSM-P2, associated with e.g., prostate cancer; and/or
(f) immunoglobulin idiotypes (associated with myeloma and B cell lymphomas, for example).

The therapeutic biomolecule may be a eukaryotic polypeptide. In one embodiment the eukaryotic polypeptide is a mammalian polypeptide. The mammalian polypeptide may be selected from the group consisting of: an enzyme; an enzyme inhibitor; a hormone; an immune system protein; a receptor; a binding protein; a transcription or translation factor; tumour growth suppressing protein; a structural protein and a blood protein.

The enzyme may be selected from the group consisting of: chymosin; gastric lipase; tissue plasminogen activator; streptokinase; a cholesterol biosynthetic or degradative steriodogenic enzyme; kinases; phosphodiesterases; methylases; de-methylases; dehydrogenases; cellulases; proteases; lipases; phospholipases; aromatases; cytochromes; adenylate or guanylaste cyclases and neuramidases.

The enzyme inhibitor may be tissue inhibitor of metalloproteinase (TIMP). The hormone may be growth hormone.

The immune system protein may be selected from the group consisting of: a cytokine; a chemokine; a lymphokine; erythropoietin; an integrin; addressin; selectin; homing receptors; T cell receptors and immunoglobulins.

The cytokine may be an interleukin, for example IL-2, IL-4 and/or IL-6, colony stimulating factor (CSF), granulocyte colony stimulating factor (G-CSF), granulocyte-macrophage colony stimulating factor (GM-CSF) or tumour necrosis factor (TNF).

The chemokine may be a macrophage inflammatory protein-2 and/or a plasminogen activator.

The lymphokine may be an interferon.

The immunoglobulin may be a natural, modified or chimeric immunoglobulin or a fragment thereof. Preferably, the immunoglobulin is a chimeric immunoglobulin having dual activity such as antibody enzyme or antibody-toxin chimera.

The hormone may be selected from the group consisting of: insulin, thyroid hormone, catecholamines, gonadotrophines, trophic hormones, prolactin, oxytocin, dopamine, bovine somatotropin, leptins; growth hormones (e.g., human grown hormone), growth factors (e.g., epidermal growth factor, nerve growth factor, insulin-like growth factor and the like).

The receptor may be a steroid hormone receptor or a peptide receptor. Preferably, the receptor is a growth factor receptor.

The binding protein may be a growth factor binding protein.

The tumour growth suppressing protein may be a protein that inhibits angiogenesis.

The structural protein may be selected from the group consisting of: collagen; fibroin; fibrinogen; elastin; tubulin; actin; and myosin.

The blood protein may be selected from the group consisting of thrombin; serum albumin; Factor VII; Factor VIII; insulin; Factor IX; Factor X; tissue plasminogen activator; protein C; von Wilebrand factor; antithrombin III; glucocerebrosidase; erythropoietin granulocyte colony stimulating factor (GCSF) or modified Factor VIII; and anticoagulants.

In one preferred embodiment, the therapeutic biomolecule is a cytokine which is capable of regulating lymphoid homeostasis, preferably a cytokine which is involved in and preferably induces or enhances development, priming, expansion, differentiation and/or survival of T cells. Thus, preferably, the cytokine is an interleukin. Most preferably, IL-2, IL-7, IL-12, IL-15, or IL-21.

The therapeutic biomolecule may be protein that is capable of enhancing reprogramming of somatic cells to cells having stem cell characteristics.

The protein that is capable of enhancing reprogramming of somatic cells to cells having stem cell characteristics may be selected from the group consisting of: OCT4, SOX2, NANOG, LIN28, p53, ART-4, BAGE, ss-catenin/m, Bcr-abL CAMEL, CAP-1, CASP-8, CDC27/m, CD 4/m, CEA, CLAUDIN-12, c-MYC, CT, Cyp-B, DAM, ELF2M, ETV6-AML1, G250, GAGE, GnT-V, GaplOO, HAGE, HER-2/neu, HPV-E7, HPV-E6, HAST-2, hTERT (or hTRT), LAGE, LDLR/FUT, MAGE-A, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Plac-1, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

Preferably MAGE-A is selected from the group consisting of: MAGE-A 1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A 12

Preferably, the protein that is capable of enhancing reprogramming of somatic cells to cells having stem cell characteristics is OCT4, SOX2, LF4; c-MYC; NANOG; LIN28.

The therapeutic biomolecule may be a biomolecule that is utilised for the modification of cells ex vivo for cell-therapy indications. Thus, preferably, the therapeutic biomolecule may be selected from the group consisting of an immunoglobulin, a T-cell receptor and NK receptor.

The therapeutic biomolecule may be an RNA molecule that is capable of regulating expression of endogenous host genes, for example an interfering RNA such as small RNAs, siRNA or microRNAs.

The composition may comprise one or more additives. These additives may be selected from a group consisting of: buffering substances; saccharides; stabilizers; cyroprotectants; lyoprotectants and chelating agents.

The compositions may be a frozen, lyophilized or sprayed dried composition. Preferably, the composition may comprise a cryoprotectant and/or lyoprotectant, for example a disaccharide such as trehalose, sucrose, maltose, or polysaccharide such as dextran or pullulan.

The compositions may further comprise a JAK/STAT pathway inhibitor that is capable of disrupting potential innate responses to formulated nucleic acid, preferably saRNA. The JAK/STAT pathway inhibitor may be selected from a group consisting of: Ruxolitinib; Tofacitinib; Oclacitinib; Momelotinib; Baricitinib; Filgotinib; Itacitinib and similar inhibitors of JAK/STAT signalling that would be known to those skilled in the art. The inventors believe comprising a polymeric composition and saRNA is novel and inventive per se.

In accordance with a third aspect, there is provided a polymeric composition comprising self-amplifying RNA (saRNA) and a plurality of polymers of formula (I):

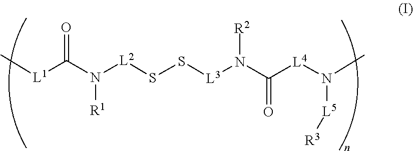

wherein L¹ to L⁵ are each independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms;
L⁶ and L⁷ are independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene or an optionally substituted 5 to 10 membered heteroarylene, wherein adjacent carbon atoms in the alkylene, alkenylene or alkynylene are optionally interrupted by one or more heteroatoms;
R¹ and R² are each independently H, an optionally substituted C₁₋₁₂ alkyl, an optionally substituted C₂₋₁₂ alkenyl or an optionally substituted C₂₋₁₂ alkynyl;
R³ is —OR⁴, —COOR⁴, —SO₂OR⁴, (OCH₂CH₂)ₘOH, or NR⁴R⁵,
R⁴ and R⁵ are each independently H, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl, wherein adjacent carbon atoms in the alkyl, alkenyl or alkenyl are optionally interrupted by one or more heteroatoms; and
m is an integer between 1 and 10;
or a pharmaceutically acceptable complex, salt, solvate, tautomeric form or polymorphic form thereof.

The average molecular mass of the plurality of polymers of formula (I) may be less than or equal to 5 kg mol⁻¹.

Notwithstanding the above, the plurality of polymers may be as defined in relation to the first aspect.

The saRNA may be as defined in relation to the second aspect.

The weight ratio of polymeric composition and the saRNA may be between 1:1 and 200:1, more preferably between 5:1 and 150:1 or between 10:1 and 100:1, and most preferably between 20:1 and 90:1, between 30:1 and 80:1, between 40:1 and 70:1 or between 45:1 and 60:1.

In accordance with a fourth aspect, there is provided a nanoparticle comprising the composition of matter as defined in the second aspect or third aspect.

Advantageously, the inventors have found that the nanoparticle of the fourth aspect enhances transfection efficiency of nucleic acids in vitro.

The nanoparticle preferably has a hydrodynamic diameter (Dn) of less than 1,000 nm or less than 750 nm, more preferably less than 500 nm, less than 400 nm or less than 200 nm, and most preferably less than 150 nm, less than 100 nm, less than 80 nm or less than 75 nm.

The nanoparticle preferably has a hydrodynamic diameter (Dn) of between 1 and 1,000 nm or between 10 and 750 nm, more preferably between 20 and 500 nm, between 30 and 400 nm or between 40 and 200 nm, and most preferably between 50 and 150 nm, between 55 and 100 nm, between 60 and 80 nm or between 65 and 75 nm.

The hydrodynamic diameter may be calculated using dynamic light scattering (DLS). The DLS measurements may be taken when the nanoparticle is disposed in a buffer solution (20 Mm HEPES, 5 wt % glucose in water, pH 7.4), and measured using the Zetasizer Nano ZS instrument. The scattering angle may be fixed at 173°. Data processing may be carried out using cumulant analysis of the experimental correlation function and the Stokes-Einstein equation may be used to calculate the hydrodynamic radii.

In a fifth aspect, there is provided a pharmaceutical composition comprising the polymeric composition of the first aspect, the composition of matter of the second or third aspect or the nanoparticle of the fourth aspect, and a pharmaceutically acceptable vehicle.

The polymeric compositions, nanoparticles and pharmaceutical compositions described herein may provide an effective means of vaccinating a subject against viral infection and cancer.

Accordingly, in a sixth aspect there is provided a vaccine comprising the polymeric composition of the first aspect, the composition of matter of the second or third aspect, the nanoparticle of the fourth aspect or the pharmaceutical composition of the fifth aspect.

Preferably, the vaccine comprises a suitable adjuvant.

The adjuvant may be an encoded molecular adjuvant that is encoded in the nucleic acid, as defined in relation to the second aspect, or as adjuvant incorporated into a delivery formulation.

The encoded molecular adjuvant may encode a cytokine, for example IL-2, IL-12, IL-21, GM-CSF, IFN-g, CCL20, CCL21, CXCL8, CXCL10, CXCL12 or an effector protein such as CD40L, Flt-3 or microbial protein, e.g flagellin or cholera toxin B.

The adjuvant incorporated into a delivery formulation may be selected from the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Saponins (Quil-A, QS-21, Tomatine, ISCOM, ISCOMATRIX™), squalene based emulsions, Carbopol, lipid nanoparticles and bacterial toxins (CT, LT).

In accordance with a seventh aspect, there is provided the polymeric composition of the first aspect, the composition of matter of the second or third aspect, the nanoparticle of the fourth aspect, the pharmaceutical composition according to the fifth aspect, or the vaccine according to the sixth aspect, for use in therapy.

In accordance with an eighth aspect, there is provided the polymeric composition of the first aspect, the composition of matter of the second or third aspect, the nanoparticle of the fourth aspect, the pharmaceutical composition according to the fifth aspect, or the vaccine according to the sixth aspect, for use in stimulating an immune response in a subject.

The immune response may be stimulated against a helminth, protozoa, bacterium, virus, fungus or cancer as per the antigens defined in the second aspect.

In accordance with a ninth aspect, there is provided the polymeric composition of the first aspect, the composition of matter of the second or third aspect, the nanoparticle of the fourth aspect, the pharmaceutical composition according to the fifth aspect, or the vaccine according to the sixth aspect, for use in the prevention, amelioration or treatment of a helminth, protozoan, fungal, bacterial or viral infection.

The helminth, protozoan, fungal, bacterial or viral infection to be treated may be an infection of a helminth, protozoa, fungus, bacterium or virus as defined in relation to the second aspect.

In accordance with a tenth aspect, there is provided the polymeric composition of the first aspect, the composition of matter of the second or third aspect, the nanoparticle of the fourth aspect, the pharmaceutical composition according to the fifth aspect, or the vaccine according to the sixth aspect, for use in the prevention, amelioration or treatment of cancer.

The cancer may be as defined in relation to the second aspect.

In accordance with an eleventh aspect of the invention, there is provided a method for treating a helminth, protozoan, fungal, bacterial or viral infection, the method comprising administering, or having administered, to a subject in need thereof, a therapeutically effective amount of the polymeric composition of the first aspect, the composition of matter of the second or third aspect, the nanoparticle of the fourth aspect, the pharmaceutical composition according to the fifth aspect, or the vaccine according to the sixth aspect.

The helminth, protozoan, fungal, bacterial or viral infection to be treated may be an infection of a protozoa, fungus, bacterium or virus as defined in relation to the second aspect.

In accordance with a twelfth aspect of the invention, there is provided a method for treating cancer, the method comprising administering, or having administered, to a subject in need thereof, a therapeutically effective amount of the polymeric composition of the first aspect, the composition of matter of the second or third aspect, the nanoparticle of the fourth aspect, the pharmaceutical composition according to the fifth aspect, or the vaccine according to the sixth aspect.

The cancer to be treated may be as defined in relation to the second aspect.

According to a thirteenth aspect, there is provided the polymeric composition of the first aspect, the composition of matter of the second or third aspect, the nanoparticle of the fourth aspect or the pharmaceutical composition according to the fifth aspect, for use in stem cell therapy.

Stem cell therapy may relate to the reprogramming somatic cells to cells having stem cell characteristics.

Somatic cells may be reprogrammed by delivering one or more proteins that are capable of enhancing reprogramming of somatic cells to cells having stem cell characteristics as defined in relation to the second aspect.

According to a fourteenth aspect, there is provided a method of modifying a cell ex vivo or in vitro, comprising delivering, to the cell, the polymeric composition of the first aspect, the composition of matter of the second or third aspect, the nanoparticle of the fourth aspect or the pharmaceutical composition according to the fifth aspect.

Preferably, the method is performed ex vivo. Preferably, the method is performed in vitro.

The cell may be a eukaryotic or prokaryotic cell. Preferably, the cell is a eukaryotic cell. More preferably, the cell is a mammalian host cell. Most preferably the cell is a human cell.

Preferably, the modified cell is suitable for cell-therapy indications.

In a fifteenth aspect, there is provided a modified cell obtained from, or obtainable by, the method of the fourteenth aspect.

Thus, according to a sixteenth aspect there is provided the modified cell of the fifteenth aspect, for use in therapy, optionally cell therapy.

It will be appreciated that the polymeric composition of the first aspect, the composition of matter of the second or third aspect, the nanoparticle of the fourth aspect, the pharmaceutical composition according to the fifth aspect, or the vaccine according to the sixth aspect (herein known as the active agents) may be used in a medicament, which may be used as a monotherapy (i.e. use of the active agent), for treating, ameliorating, or preventing disease or vaccination. Alternatively, the active agents according to the invention may be used as an adjunct to, or in combination with, known therapies for treating, ameliorating, or preventing disease.

The polymeric composition, nanoparticle, pharmaceutical composition, or vaccine of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles (with RNA on the surface or encapsulated) or any other suitable form that may be administered to a person or animal in need of treatment or vaccination. It will be appreciated that the vehicle of medicaments according to the invention should be one which is well-tolerated by the subject to whom it is given.

The polymeric composition, nanoparticle, pharmaceutical composition, or vaccine of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the polymeric composition, nanoparticle, pharmaceutical composition, or vaccine is required and which would normally require frequent administration (e.g. at least daily injection).

In a preferred embodiment, however, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion), or intramuscular (bolus or infusion).

It will be appreciated that the amount of polymeric composition, nanoparticle, pharmaceutical composition, or vaccine that is required is determined by its biological activity and bioavailability, which in turn depends on the mode of administration, the physiochemical properties of the polymeric composition, nanoparticle, pharmaceutical composition, or vaccine and whether it is being used as a monotherapy or in a combined therapy. The frequency of administration will also be influenced by the half-life of the active agent within the subject being treated. Optimal dosages to be administered may be determined by those skilled in the art, and will vary with the particular polymeric composition, nanoparticle, pharmaceutical composition, or vaccine in use, the strength of the pharmaceutical composition, the mode of administration, and the type and advancement of the viral infection. Additional factors depending on the particular subject being treated will result in a need to adjust dosages, including subject age, weight, sex, diet and time of administration.

Generally, a daily dose of between 0.001 μg/kg of body weight and 10 mg/kg of body weight, or between 0.01 μg/kg of body weight and 1 mg/kg of body weight, of the polymeric composition, nanoparticle, pharmaceutical composition, or vaccine of the invention may be used for treating, ameliorating, or preventing a disease, depending upon the active agent used.

Daily doses may be given as a single administration (e.g. a single daily injection or inhalation of a nasal spray). Alternatively, the polymeric composition, nanoparticle, pharmaceutical composition, or vaccine may require administration twice or more times during a day. As an example, the polymeric composition, nanoparticle, pharmaceutical composition, or vaccine may be administered as two (or more depending upon the severity of the disease being treated) daily doses of between 0.07 g and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the polymeric composition, nanoparticle, pharmaceutical composition, or vaccine according to the invention to a patient without the need to administer repeated doses.

Preferably, however, the polymeric composition, nanoparticle, pharmaceutical composition, or vaccine according to the invention may be given as a weekly dose, and more preferably a fortnightly dose. Alternatively, the polymeric composition, nanoparticle, pharmaceutical composition, or vaccine may be given as a single dose.

Known procedures, such as those conventionally employed by the pharmaceutical industry (e.g. in vivo experimentation, clinical trials, etc.), may be used to form specific formulations of the polymeric composition, nanoparticle, pharmaceutical composition, or vaccine according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the polymeric composition, nanoparticle, pharmaceutical composition, or vaccine is any amount which, when administered to a subject, is the amount of the aforementioned that is needed to ameliorate, prevent or treat any given disease.

For example, the polymeric composition, nanoparticle, pharmaceutical composition, or vaccine of the invention may be used may be from about 0.0001 mg to about 800 mg, and preferably from about 0.001 mg to about 500 mg. It is preferred that the amount of polymeric composition, nanoparticle, pharmaceutical composition, or vaccine is an amount from about 0.01 mg to about 250 mg, and most preferably from about 0.01 mg to about 1 mg. Preferably, the polymeric composition, nanoparticle, pharmaceutical composition, or vaccine according to the invention is administered at a dose of 1-200 μg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. polymeric composition, nanoparticle, pharmaceutical composition, or vaccine according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The polymeric composition, nanoparticle, pharmaceutical composition, or vaccine according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The polymeric composition, nanoparticle, pharmaceutical composition, or vaccine of the invention may be prepared as a sterile solid composition that may be dissolved or suspended at the time of administration using sterile water, saline, or other appropriate sterile injectable medium.

The polymeric composition, nanoparticle, pharmaceutical composition, or vaccine of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The polymeric composition, nanoparticle, pharmaceutical composition, or vaccine according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

In accordance with a seventeenth aspect, there is provided a method of producing a high molar mass poly(amido amine), the method comprising contacting a compound of formula (II):

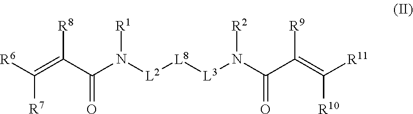

with a compound of formula (III):

NHR¹²R¹³ (III)

in the presence of a Lewis base to thereby cause the compounds of formula (II) and formula (III) to undergo a polymerisation reaction and produce the high molar mass poly(amido amine);
wherein R¹ and R² are each independently H, an optionally substituted C₁₋₁₂ alkyl, an optionally substituted C₂₋₁₂ alkenyl or an optionally substituted C₂₋₁₂ alkynyl;
R⁶ to R¹¹ are each independently H, a halogen, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl;
L² and L³ are each independently absent or an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms;
L⁸ is absent or is —S—S—;
R¹² is an optionally substituted C₁₋₁₂ alkyl, an optionally substituted C₂₋₁₂ alkenyl, an optionally substituted C₂₋ₘ alkynyl, an optionally substituted C₃₋₆ cycloalkyl, an optionally substituted 3 to 8 membered heterocyclyl, an optionally substituted C₆₋₁₂ aryl, an optionally substituted 5 to 10 membered heteroaryl, wherein adjacent carbon atoms in the alkyl, alkenyl or alkenyl are optionally interrupted by one or more heteroatoms; and R¹³ is H, an optionally substituted C₁₋₁₂ alkyl, an optionally substituted C₂₋₁₂ alkenyl, an optionally substituted C₂₋ₘ alkynyl, an optionally substituted C₃₋₆ cycloalkyl, an optionally substituted 3 to 8 membered heterocyclyl, an optionally substituted C₆₋₁₂ aryl, an optionally substituted 5 to 10 membered heteroaryl, wherein adjacent carbon atoms in the alkyl, alkenyl or alkenyl are optionally interrupted by one or more heteroatoms.

The method of the seventeenth aspect may be used to produce a compound according to the first aspect.

Preferably, R¹ and R² are as defined in relation to the first aspect.

Preferably, R⁶ to R¹¹ are each independently H, a halogen, an optionally substituted C₁₋₃ alkyl, an optionally substituted C₂₋₃ alkenyl or an optionally substituted C₂₋₃ alkenyl. More preferably, R⁶ to R¹¹ are each H.

Preferably, L² and L³ are as defined in relation to the first aspect.

Preferably, L⁸ is —S—S—.

The compound of formula (II) may be a compound of formula (IIa):

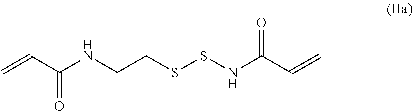

Accordingly, the compound of formula (II) may be N,N′-cystaminebisacrylamide (CBA).

Preferably, R¹² is an optionally substituted C₂₋₈ alkyl, an optionally substituted C₂₋₈ alkenyl or an optionally substituted C₂₋₈ alkynyl. More preferably R¹² is an optionally substituted C₃₋₅ alkyl, an optionally substituted C₃₋₅ alkenyl or an optionally substituted C₃₋₅ alkynyl.

The alkyl, alkenyl or alkynyl is preferably substituted. Preferably, the alkyl, alkenyl or alkynyl is substituted with an OR¹⁴ group. Preferably, R¹⁴ is H.

Preferably, R¹³ is H, an optionally substituted C₁₋₆ alkyl, an optionally substituted C₂₋₆ alkenyl or an optionally substituted C₂₋₆ alkynyl. More preferably, R¹³ is H, an optionally substituted C₁₋₃ alkyl, an optionally substituted C₂₋₃ alkenyl or an optionally substituted C₂₋₃ alkynyl. Most preferably, R¹³ is H.

Accordingly, the compound of formula (III) may be 4-amino-1-butanol (ABOL).

The Lewis base may be ammonia or an amine. The amine may be a primary amine, a secondary amine or a tertiary amine. The tertiary amine may be trimethyl amine or triethyl amine. Most preferably, the Lewis base is triethyl amine (TEA).

The molar ratio of the compound of formula (II) to the compound of formula (III) is preferably between 1:1 and 1,000:1, more preferably is between 2:1 and 100:1, between 3:1 and 75:1 or between 4:1 and 50:1, and most preferably is between 5:1 and 25:1, between 6:1 and 20:1, between 7:1 and 15:1, between 8:1 and 12:1 or between 9:1 and 11:1. In a preferred embodiment, the molar ratio of the compound of formula (II) to the compound of formula (III) is about 10:1.

The molar ratio of the compound of formula (II) to the Lewis base is preferably between 1:1 and 1,000:1, more preferably is between 10:1 and 1,000:1, between 25:1 and 750:1 or between 50:1 and 500:1, and most preferably is between 60:1 and 250:1, between 70:1 and 200:1, between 80:1 and 150:1, between 90:1 and 120:1 or between 95:1 and 110:1. In a preferred embodiment, the molar ratio of the compound of formula (II) to the Lewis base is about 100:1.

The molar ratio of the compound of formula (III) to the Lewis base is preferably between 1:1 and 1,000:1, more preferably is between 2:1 and 100:1, between 3:1 and 75:1 or between 4:1 and 50:1, and most preferably is between 5:1 and 25:1, between 6:1 and 20:1, between 7:1 and 15:1, between 8:1 and 12:1 or between 9:1 and 11:1. In a preferred embodiment, the molar ratio of the compound of form formula (III) to the Lewis base is about 10:1.

The compounds of formula (II) and (III) may be contacted in a first solvent, to create a reactive solution. The first solvent may comprise an alcohol and/or water. The alcohol may be a C₁₋₆ alcohol, and is preferably methanol or ethanol, and most preferably is methanol.

In a preferred embodiment, the first solvent comprises an alcohol and water. The volumetric ratio of the alcohol to water may be between 1:5 and 50:1, more preferably between 1:2 and 25:1 or between 1:1 and 10:1, and most preferably is between 2:1 and 8:1 or between 3:1 and 5:1. In a preferred embodiment, the ratio of alcohol to water is about 4:1.

The concentration of the compound of formula (II) in the solvent may be between 0.1 and 50 M, more preferably is between 0.5 and 25 M or between 1 and 10 M, and most preferably is between 2 and 8M or between 4 and 6 M.

The concentration of the compound of formula (III) in the solvent may be between 0.01 and 5 M, more preferably is between 0.05 and 2.5 M or between 0.1 and 1 M, and most preferably is between 0.2 and 0.8 M or between 0.4 and 0.6 M.

The concentration of the Lewis base in the solvent may be between 0.001 and 0.5 M, more preferably is between 0.005 and 0.25 M or between 0.01 and 0.1 M, and most preferably is between 0.02 and 0.08 M or between 0.04 and 0.06 M.

The compounds of formula (II) and (III) may be contacted at a temperature of between 0 and 100° C., more preferably between 5 and 90° C., between 10 and 80° C. or between 20 and 70° C., and most preferably between 30 and 6° C. or between 40 and 50° C.

The compounds of formula (II) and (III) may be contacted in the dark.

The compounds of formula (II) and (III) may be contacted for between 1 hour and 50 days, more preferably between 1 and 25 days or between 2 and 20 days, and most preferably between 3 and 18 days, between 4 and 15 days or between 5 and 14 days. It may be appreciated that the exact amount of time may vary depending upon the desired molecular mass of the polymer.

Subsequent to contacting the compounds of formula (II) and (III), the method may comprise stopping the reaction. The method may comprise stopping the reaction by quenching the reaction solution with a second solvent. The second solvent may be an alcohol or water. The alcohol may be a C₁₋₆ alcohol, and is preferably methanol or ethanol, and most preferably is methanol.

The volumetric ratio of the first solvent to the second solvent may be between 1:1 and 1:10,000, more preferably between 1:10 and 1:1,000 or between 1:50 and 1:750, and most preferably between 1:100 and 1:500, between 1:200 and 1:400 or between 1:250 and 1:300.

Subsequent to contacting the compounds of formula (II) and (III), and preferably subsequent to stopping the reaction, the method may comprise contacting the high molar mass poly(amido amine) with an acid. The acid may be any inorganic or organic acid, such as hydrochloric acid, sulfuric acid, nitric acid or acetic acid.

The acid may be added at a concentration of between 0.01 and 100 M, more preferably between 0.05 and 50 M or between 0.1 and 10 M, and most preferably between 0.2 and 7.5 M, between 0.5 and 5 M, between 0.75 and 2.5 M or between 0.9 and 1.5 M.

The method may comprise purifying the high molar mass poly(amido amine). The high molar mass poly(amido amine) may be purified using dialysis, preferably against acidic water.

In accordance with an eighteenth aspect, there is provided a method of producing a composition of matter or a nanoparticle, the method comprising contacting a polymeric composition as defined in the first aspect with a nucleic acid.

The method may comprise producing the composition of matter of the second or third aspect or the nanoparticle of the fourth aspect.

The nucleic acid may be as defined in relation to the second aspect.

The weight ratio of polymeric composition to the nucleic acid may be between 1:1 and 200:1, more preferably between 5:1 and 150:1 or between 10:1 and 100:1, and most preferably between 20:1 and 90:1, between 30:1 and 80:1, between 40:1 and 70:1 or between 45:1 and 60:1.

The method may comprise contacting a first solution, comprising the polymeric composition, with a second solution, comprising the nucleic acid.

The first solution may comprise a solvent. The solvent may comprise water and/or a buffer. In a preferred embodiment, the solvent comprises water and a buffer. The buffer may be configured to maintain a pH between 5 and 9 at 20° C., more preferably a pH of between 6 and 8 or between 7 and 7.8 at 20° C., and most preferably a pH between 7.2 and 7.6 at 20° C. The buffer may be a HEPES buffer.

The first solution may comprise the polymeric composition at a concentration of between 0.0001 and 500 μg/μL, more preferably between 0.001 and 100 μg/μL or between 0.01 and 50 μg/μL, and most preferably between 0.05 and 10 μg/μL, between 0.1 and 1 μg/μL or between 0.2 and 0.5 μg/μL.

The second solution may comprise a solvent. The solvent may comprise water and/or a buffer. In a preferred embodiment, the solvent comprises water and a buffer. The buffer may be configured to maintain a pH between 5 and 9 at 20° C., more preferably a pH of between 6 and 8 or between 7 and 7.8 at 20° C., and most preferably a pH between 7.2 and 7.6 at 20° C. The buffer may be a HEPES buffer.

The second solution may comprise the nucleic acid at a concentration of between 0.00001 and 10 μg/μL, more preferably between 0.0001 and 1 μg/μL or between 0.0005 and 0.5 μg/μL, and most preferably between 0.0008 and 0.05 μg/μL or between 0.001 and 0.005 μg/μL.

The volumetric ratio of the first solution to the second solution may be between 10:1 and 1:20, more preferably between 2:1 and 1:10 or between 1:1 and 1:8, and most preferably is between 1:2 or 1:6 or between 1:3 and 1:5.

The second solution may be added to the first solution over a period of time. The first solution may be stirred constantly as the second solution is added thereto. The second solution may be added to the first solution at an approximately constant rate. The period of time may be between 1 second and 24 hours, more preferably between 10 seconds and 1 hour or between 30 seconds and 30 minutes, and most preferably is between 1 and 20 minutes, 2 and 10 minutes, 3 and 8 minutes or 4 and 6 minutes.

Advantageously, by adding the second solution over a period of time the inventors were able to obtain nanoparticles with diameters of about 70 nm.

In a nineteenth aspect, there is provided a process for making the pharmaceutical composition according to the fifth aspect, the method comprising contacting the polymeric composition of the first aspect, the composition of matter of the second or third aspect or the nanoparticle of the fourth aspect, with a pharmaceutically acceptable vehicle.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

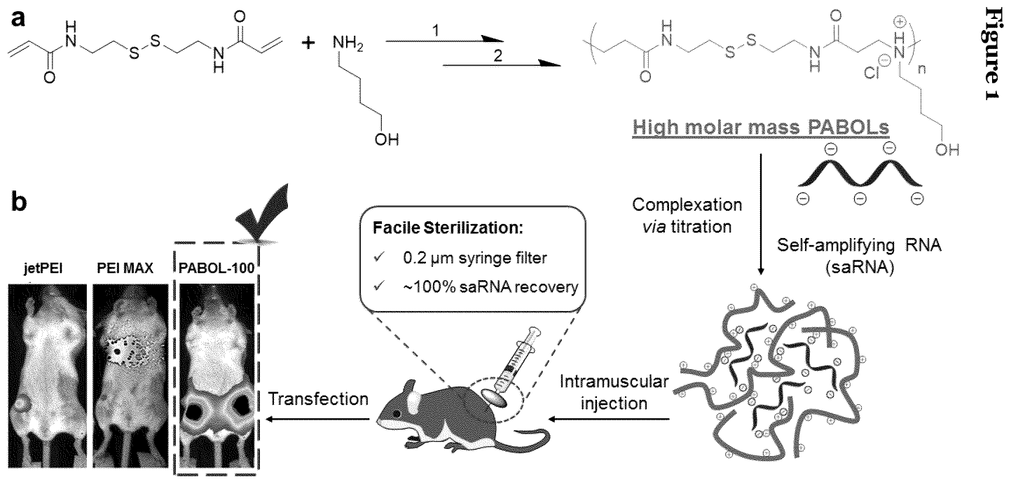

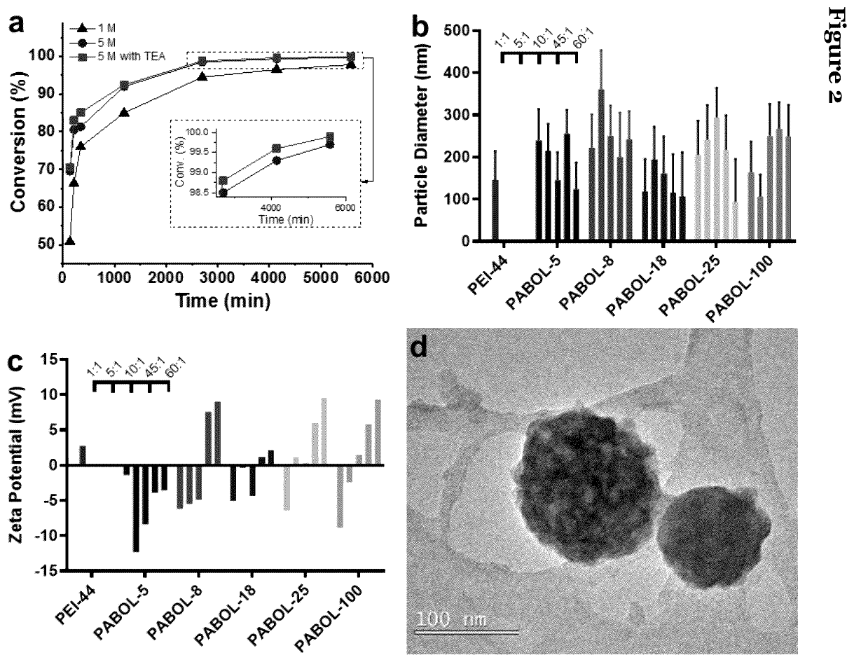

FIG. 1 is a schematic illustration of (a) improved Aza-Michael addition to afford high molar mass poly(amido amine)s, poly(CBA-4-amino-1-butanol)s pABOLs with molar masses up to 167 kg mol¹; (b) complexation with self-amplifying RNA (saRNA) via titration method and transfection efficacy of the pABOL-100 polyplexes, comparing to jetPEI and PEI MAX;

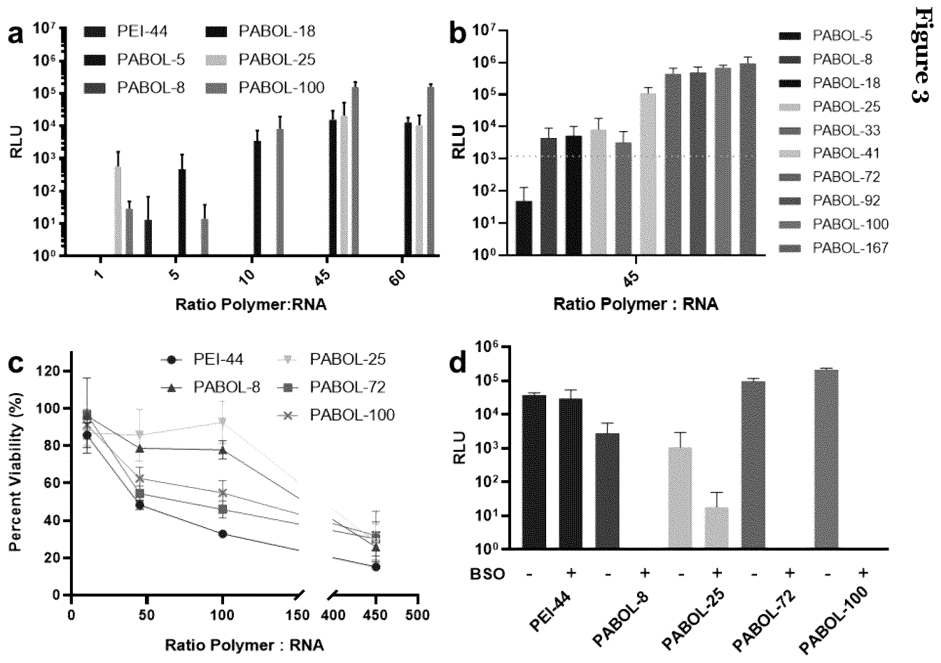

FIG. 2 shows the synthesis of high MW pABOL and characterization of resulting saRNA polyplexes. (a) Polymerization kinetics of ABOL with CBA under different reaction conditions. The conversion values were calculated from the integrals of doubled bond signals at 5.60˜6.23 ppm, using the methylene signals at 1.36˜1.47 ppm as the internal reference; (b) and (c) particle diameter and zeta potential of polyplexes form via the direct mixing method between pABOLs and saRNA at polymer to saRNA weight ratio ranging from 1 to 45. PEI-44 [poly(ethylene imine), linear, 44 kg mol⁻¹] at weight ratio of 1 was used as the reference; and (d) typical TEM of polyplexes (pABOL-100/saRNA=45:1, w/w) stained with 2 wt % uranyl acetate (scale bar: 100 nm);

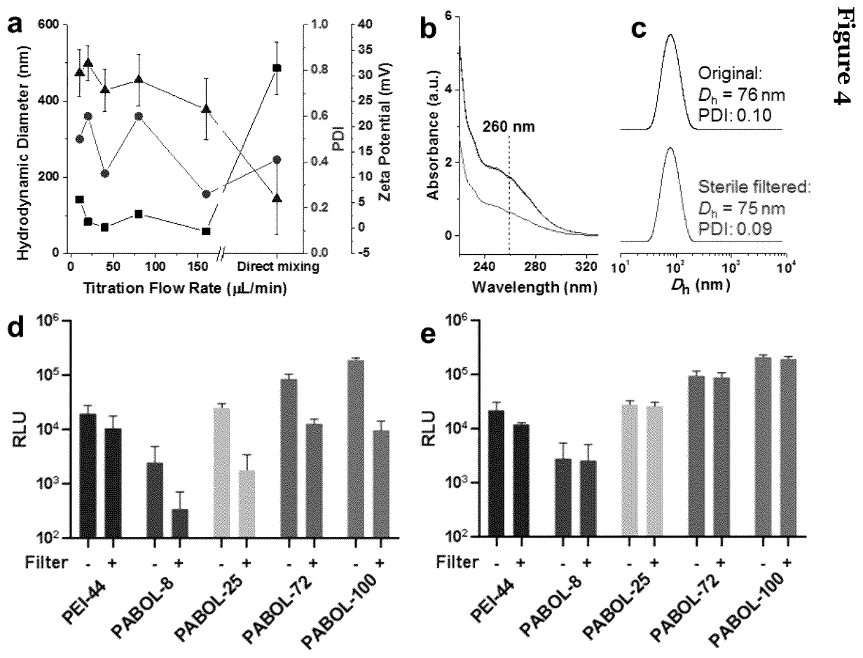

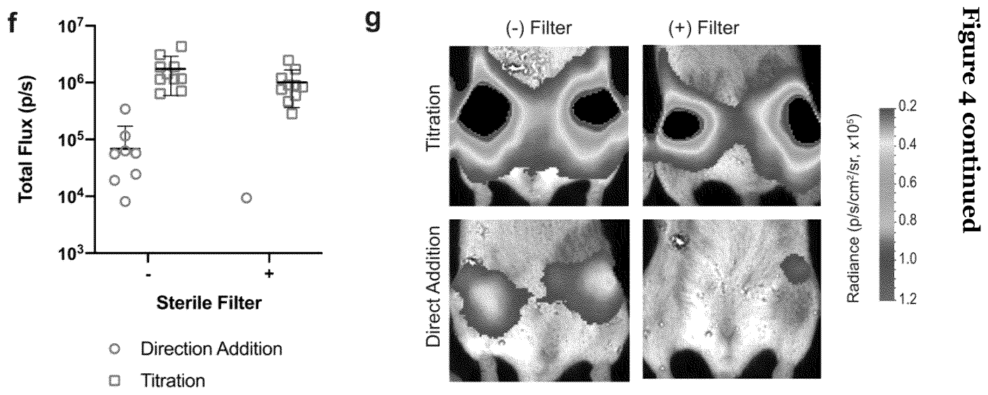

FIG. 3 shows the in vitro transfection efficiency and cytotoxicity of pABOL polyplexes. (a) Quantification of fLuc expression in relative light units (RLU) of polyplexes formed by PEI-44 and pABOLs with saRNA, 24 h after transfection at mass ratios ranging from 1:1 to 45:1 (w/w); (b) quantification of fLuc expression in RLU of polyplexes formed by all pABOLs in Table 1 at a mass ratio of 45:1 (see Figure S13 for other mass ratios); (c) cytotoxicity studies of polyplexes formed at mass ratios ranging from 10:1 to 450:1 (saRNA loading=100 ng), 24 h after initial transfection; and (d) quantification of fLuc expression in RLU of polyplexes, using untreated cells (−) and cells (+) treated with Glutathione (GSH) inhibitor, Buthionine sulphoximine (BSO);

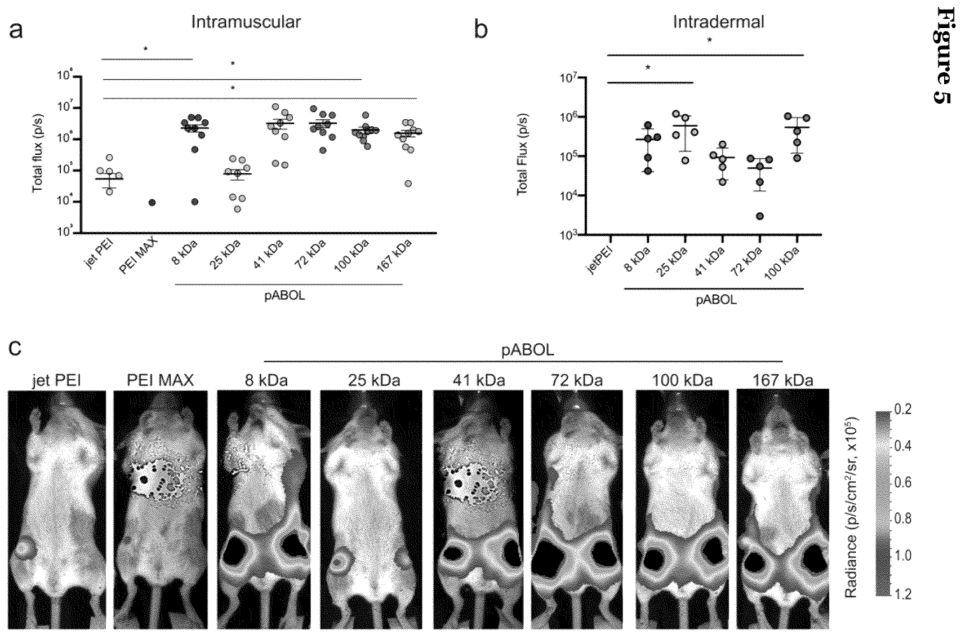

FIG. 4 shows functional characterization of polyplexes prepared using either the direct addition or titration methods in vitro and in vivo. (a) Hydrodynamic diameter, polydispersity and zeta potential of polyplexes form via the titration method (adding saRNA to polymer) at various titration flow rates (from 10 to 160 μL min⁻¹), using pABOL-100 (polymer/RNA=45:1, w/w, see SI for details). Data from direct mixing method is included for reference; (b) absorbance curves of polyplexes measured by Nanodrop before (black) and after (red) passing through a 0.2 m syringe filter. (pink curve: absorbance of pABOL-100 in buffer with the same concentration as in the obtained polyplex solution); (c) hydrodynamic diameter of polyplexes measured by DLS before (black) and after (red) passing through a 0.2 μm syringe filter; (d) and (e) quantification of fLuc expression in RLU, using filtered (+) and non-filtered (−) polyplexes formed via the direct mixing and titration method, respectively; and (f) quantification of fLuc expression of filtered or non-filtered pABOL polyplexes formed by direct addition and titration methods, 7 d after injection. Mice were injected with 5 μg of saRNA in each leg, and a ratio polymer to RNA of 45:1 (w/w) for pABOL. Each circle represents one leg of one animal, and bar represents mean+/−SEM.; (g) Representative images of each group, corresponding to (f);

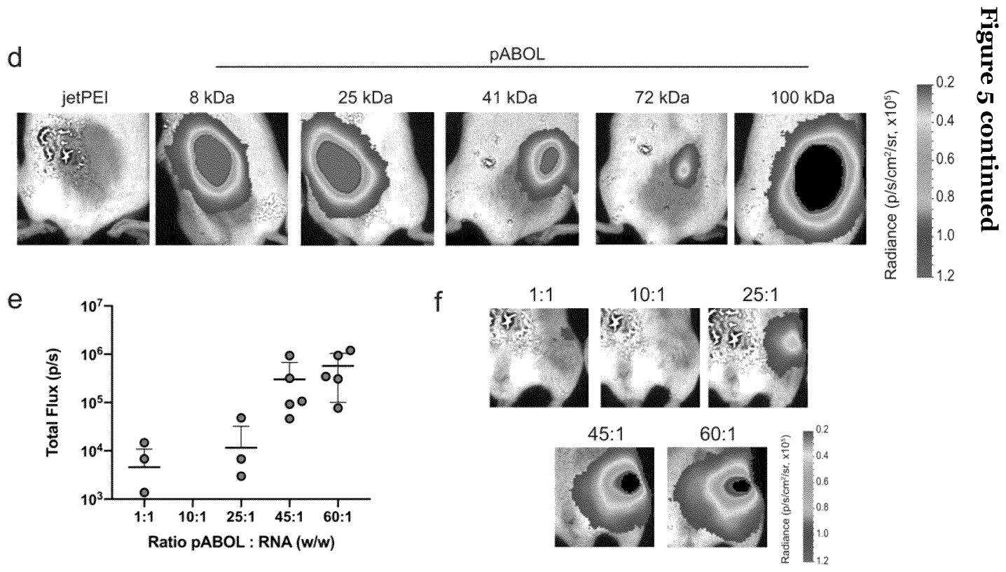

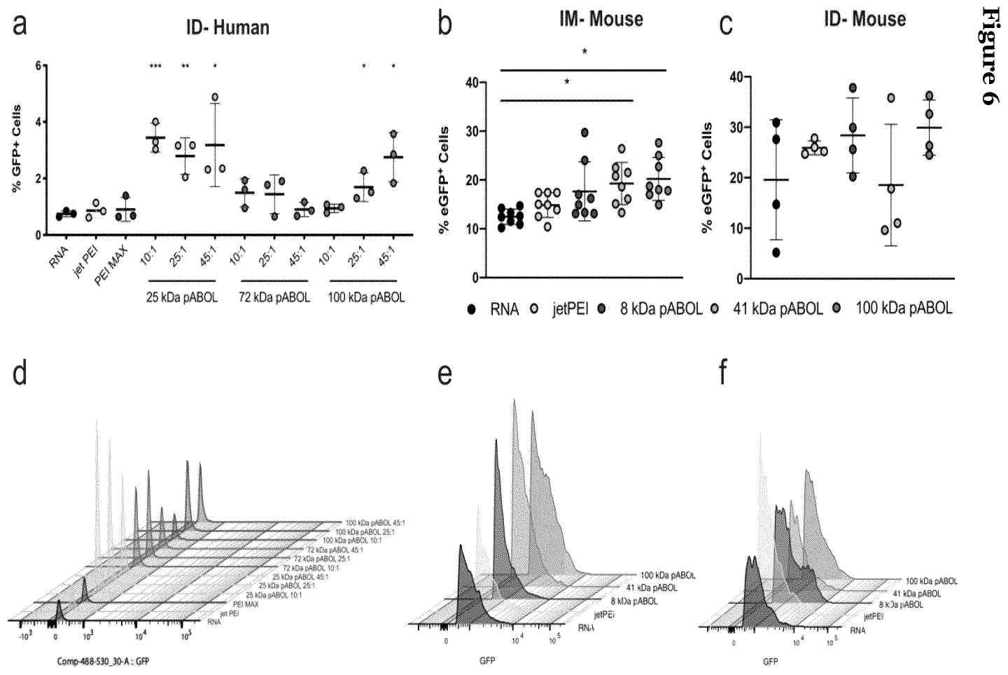

FIG. 5 shows the effect of molecular weight, route of administration and ratio of pABOL to RNA on in vivo expression of fLuciferase-encoding saRNA polyplexes. a-b) Quantification of fLuc expression of PEI (jetPEI and PEI MAX) and pABOL polyplexes in total flux (p/s), 7 d after injection. Mice were injected with 5 μg of saRNA either (a) intramusculary or (b) intradermally, and a ratio polymer to RNA of 45:1 (w/w) for pABOL, 1:1 for PEI MAX, and an N:P of 8 for jetPEI. Each circle represents one leg of one animal, and bar represents mean+/−SD. c-d) Representative images of each group. e) Quantification of fLuc expression in vivo 7 d after IM injection of 5 μg of saRNA with varying ratios of pABOL to RNA. Each circle represents one leg of one animal, and bar represents mean+/−SD. f) Representative images of each group, corresponding to e). * Indicates significance based on a one-way ANOVA with α=0.05;

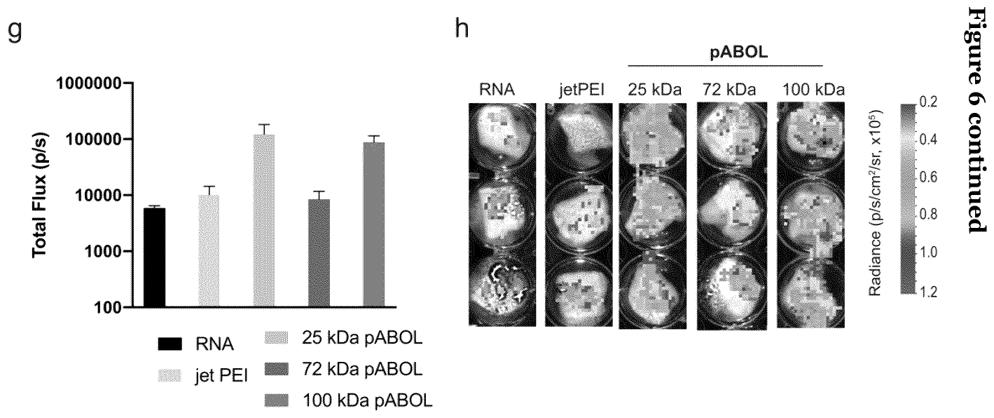

FIG. 6 shows cellular expression of saRNA after IM (mouse) or ID (human, mouse) injection with polyplex formulations. a) Percentage of eGFP+ cells out of total live cells for each formulation after an intradermal injection of 2 μg of saRNA in human skin explants. Explants were analyzed 72 h after initial injection. jetPEI and PEI MAX were formulated at ratios of N/P=8 and 1, respectively. pABOL formulations were prepared at ratios of 10:1, 25:1, and 45:1. Bars represent mean+/−SD for n=3, with *, ** and *** indicating significance of p<0.05, 0.01 and 0.001, respectively; b-c) Percentage of eGFP+ cells out of total live cells for each formulation after either IM (b) or ID (c) injection of 5 μg of saRNA in mice. Tissue was excised 7 d after initial injection. jetPEI was formulated at a ratio of N/P=8, and pABOL formulations were prepared at a ratio of 45:1. Bars mean+/−SD for n=8 (IM) and 4 (ID), with * indicating significance of p<0.05. d-f) Histograms of mean eGFP fluorescence intensity (MFI) for each formulation in human skin explants (d), IM injection in mice (e) and ID injection in mice (f). g) fLuc expression of human skin explants after ID injection with 2 μg of saRNA. Explants were analyzed 72 h after initial injection. Bars represent mean+/−SD for n=3. h) Representative images corresponding to g);

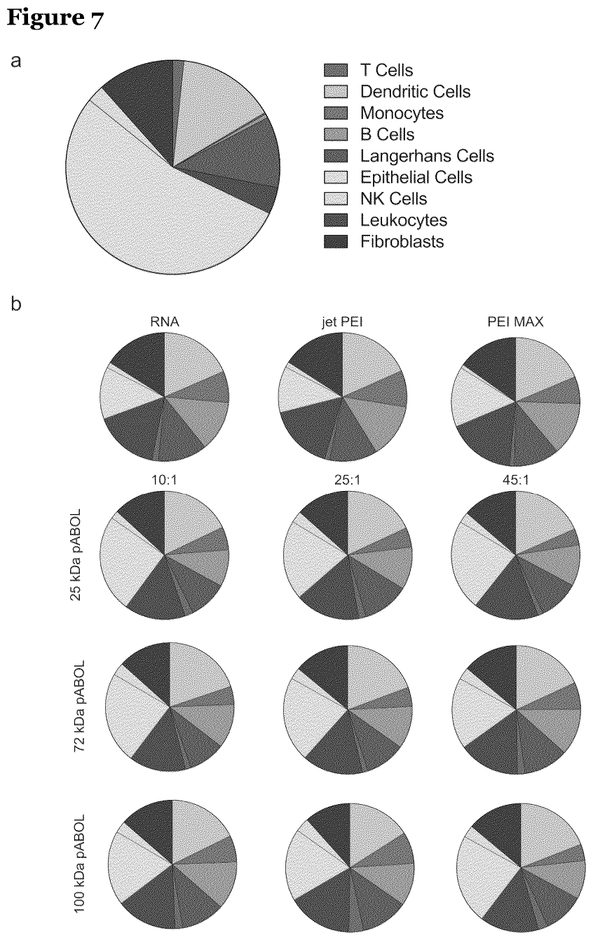

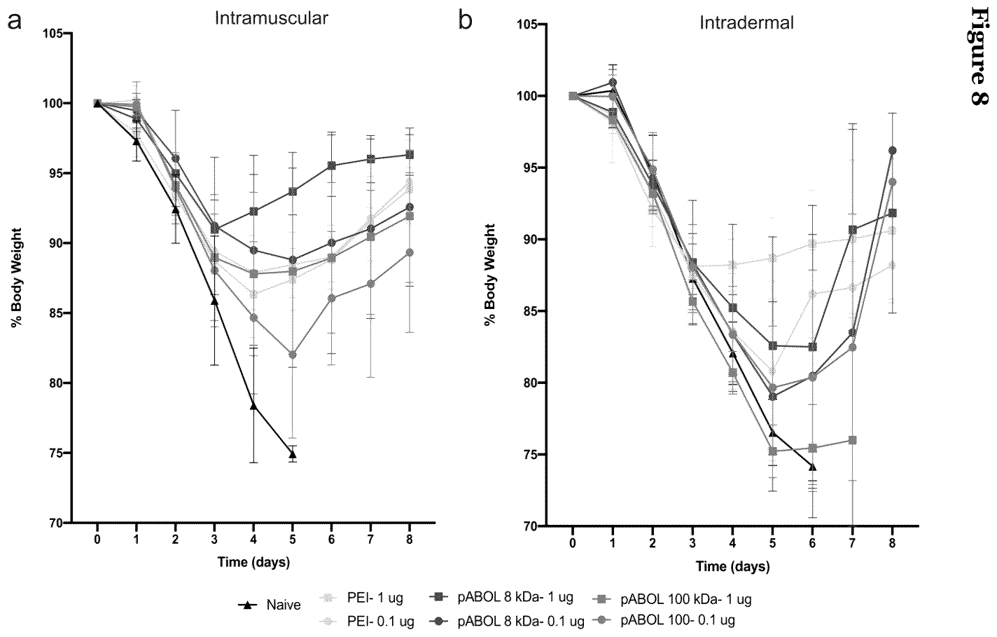

FIG. 7 shows phenotypic identity of cell present in human skin explants and GFP+ cells after intradermal (ID) injection of polyplex formulations as determined by flow cytometry. a) Identity of cells in the population of total cells extracted from human skin explants; and b) identity of GFP-expressing skin cells from explants treated with polyplex formulated eGFP-encoding saRNA. Cells identified using the following antibodies: epithelial cells (CD45−), fibroblasts (CD90+), NK cells (CD56+), leukocytes (CD45+), Langerhans cells (CD1a+), monocytes (CD14+), dendritic cells (CD11c+), T cells (CD3+) and B cells (CD19+);

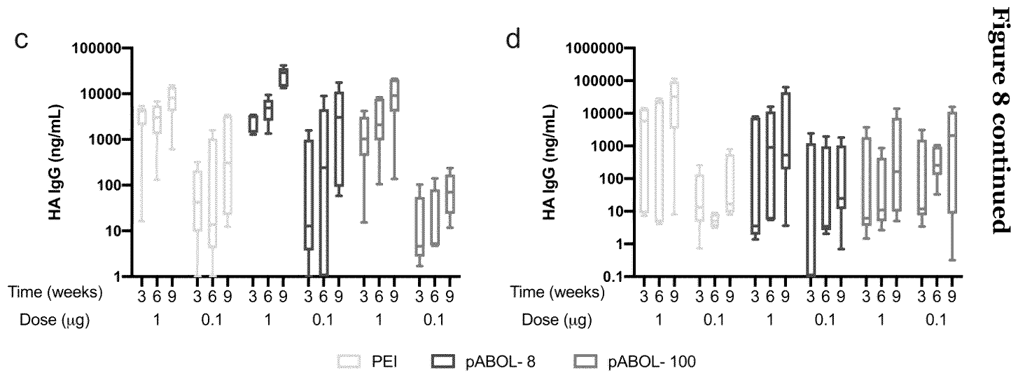

FIG. 8 shows immunogenicity of HA-encoding saRNA polyplexes. a-b) Change in body weight after IN challenge with Cal/09 flu virus for mice injected either IM (a) or ID (b). Dots represent mean percentage, normalized to Day 0 for each mouse, +/−SD for n=5; c-d) HA antigen-specific IgG antibody titers following immunization with prime and boost of saRNA complexed with jetPEI, 8 kg mol⁻¹ pABOL or 100 kg mol⁻¹ pABOL for mice injected either IM (c) or ID (d). Each bar represents mean+/−SEM for n=5 at each time point; and

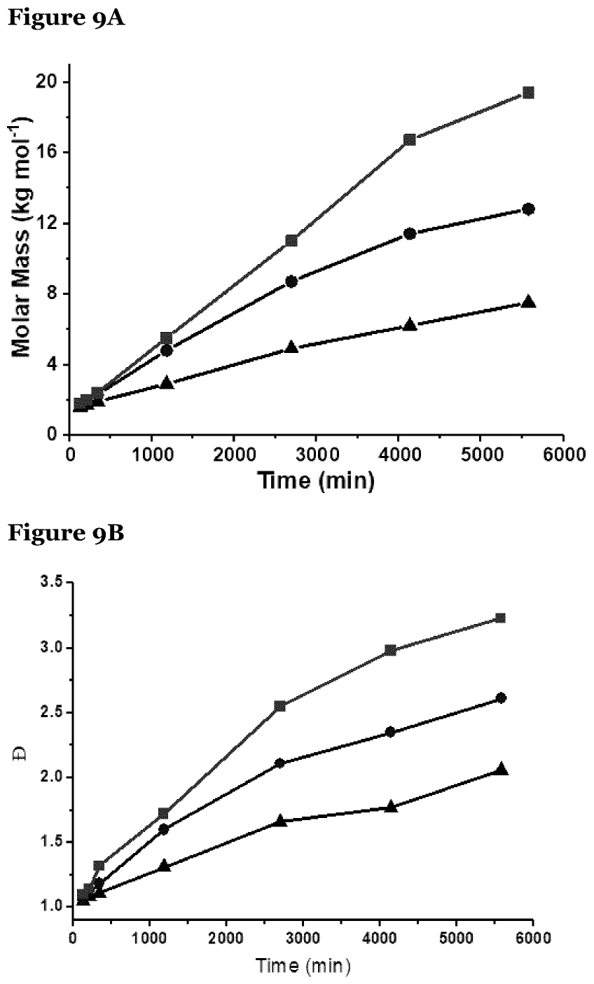

FIG. 9 shows graphs monitoring the (a) molecular weight and (b) molecular weight dispersity (Ð) over time using SEC, corresponding to 1 M (▴), 5 M (●) and 5 M with TEA (▪).

### EXAMPLES

Poly(amido amine)s (pAAs) fit the inventors' polymeric criteria and in addition, depending on the monomer combinations, linear pAAs generally have good water solubility, stability against hydrolysis and tunable degradation.¹⁶ The use of a disulphide monomer, N,N′-cystaminebisacrylamide (CBA), enables bioreduction via a disulphide backbone, which undergoes rapid cleavage intracellularly due to the presence of glutathione (GSH).¹⁶ Furthermore, preparation of pAAs is simple; two monomers are mixed together and undergo Aza-Michael polyaddition, which is a facile approach for scale-up and clinical translation. However, previous reports on pAAs reports polymers with molar mass limited to −5 kg mol⁻¹ with ˜10 repeat units,¹⁶⁻¹⁹ which to be more accurate, are just oligomers.

As explained below, the inventors prepared a library of poly(CBA-4-amino-1-butanol) (pABOL) (see FIG. 1) with varying molar mass, ranging from 5 to 167 kg mol⁻¹, using an optimized Aza-Michael polyaddition synthesis protocol. Using commercially available pEIs that have been used extensively in vitro and in vivo as a positive control,²⁰⁻²⁴ the inventors characterized the in vitro transfection efficiency and cytotoxicity. The inventors then devised a method of polyplex preparation that enables monodisperse particles and sterile filtration, which is imperative for clinical translation of this formulation. Furthermore, the inventors characterized the relationship between pABOL molar mass and protein expression in vivo using both intramuscular (IM) and intradermal (ID) injection. They then assessed whether protein expression was due to the quality or quantity of cellular expression ex vivo in human skin explants and in vivo in murine skin and muscle and phenotyped the cells in human skin that express pABOL/saRNA complexes. Finally, the inventors use pABOL and hemagglutinin (HA)-encoding saRNA as a vaccine model and observe the immunogenicity and ability to protect against influenza challenge compared to pEI in vivo.

### Example 1—Synthesis of pABOLs with High Molar Masses

First, the inventors increased the initial monomer concentration from 1.0 M to 5.0 M (defined as the CBA concentration). This led to a significant increase in reaction rate, reaching 98% of double bond conversion after 2 days with a M_{w} of 8.7 kg mol⁻¹ (FIG. 9a) in comparison with 4.9 kg mol⁻¹ (conv. =94%) observed at 1.0 M. However, due to the high viscosity the reaction hit a kinetic barrier.

To address this issue, triethylamine (TEA) was employed as a Lewis base catalyst to further increase the reaction rate. The addition of TEA increased the conversion by 0.2% in 4 days (FIG. 1) and doubled the molecular mass compared to the non-catalysed reaction (FIG. 9a). With the combination of higher monomer concentration and use of TEA as a catalyst, the targeted conversions (>99.5%) can be easily achieved within 3 days. The conversions were not monitored after 4 days as the double bond conversion exceeded 99.9% in the catalysed reaction; thus the residual signals were too weak to be detected in the NMR spectroscopy even with 1024 scans. However, higher molar masses are accessible by extending reaction period from 5 to 14 days. pABOLs with molar masses ranging from 5 to 167 kg mol⁻¹ (Table 1) were successfully prepared via the improved aza-Michael polyaddition conditions.

Thus, for the first time, the inventors were able to synthesize pABOLs with molar masses >30 kg mol⁻¹. Moreover, the method described here may enable synthesis of high molar mass poly(amido amine)s given the broad range of commercial chemicals that undergo aza-Michael polyaddition.

TABLE 1
pABOLs with variable molar masses.

| # | Polymers^{a} | M_{w} (kDa)^{b} | Ð^{b} |
|---|---|---|---|
| 0 | pABOL (ref.)^{16, 25} | ca. 5~6 | ca. 1.1~1.4 |
| 1 | pABOL-5^{c} | 5 | 1.7 |
| 2 | pABOL-8 | 8 | 2.0 |
| 3 | pABOL-18 | 18 | 2.5 |
| 4 | pABOL-25 | 25 | 2.9 |
| 5 | pABOL-33 | 33 | 4.6 |
| 6 | pABOL-41 | 41 | 3.9 |
| 7 | pABOL-72 | 72 | 5.9 |
| 8 | pABOL-92 | 92 | 5.0 |
| 9 | pABOL-100 | 105 | 6.4 |
| 10 | pABOL-167 | 167 | 4.7 |

^{a}Polymerization conditions: [CBA]/[ABOL]/[TEA] = 1.01/1/0.1, 5.0M in MeOH/H₂O (4/1, v/v) at 45° C., for 5~14 d under N₂ and in dark;
^{b}Determined by SEC, in DMF, at 30° C., calibrated using poly(methyl methacrylate) standards with narrow Ð;
^{c}Polymerized without TEA as the catalyst.

### Example 2—Increasing pABOL Molar Mass Enhances Transfection Efficiency of Nucleic Acids In Vitro

In order to assess the effect of polymer molar mass on complexation, the polyplexes were prepared via a direct mixing procedure. Given that the binding sites on both the saRNA and high molar mass pABOLs might not be completely accessible, due to the higher-order structure and the sterically hindered tertiary amine groups, respectively, the inventors opted to use a range of polymer/RNA weight ratios (from 1:1 to 60:1) instead of the commonly used N/P values. It is noteworthy that theoretical average molar masses per charge of pABOLs and saRNA are 349.5 g mol⁻¹ and 339.5 g mol⁻¹, respectively, suggesting the weight ratios are close to N/P values. When combined, pABOL and saRNA form nanoparticles with diameter ranging from 100 to 400 nm regardless of the weight ratios (FIG. 2b). However, only at weight ratios higher than 45:1 can nanoparticles exhibit sufficient positive surface charge to maintain sufficient colloidal stability as well as good cell permeability (FIG. 2c).²⁶ The inventors observed that a polymer/RNA weight ratio of at least 10:1 (N/P=8) was required to reach a neutral surface charge, which confirms their assumption that due to the higher-order structure and steric hindrance a certain amount of binding sites are left unbound during the polyplex formation. Furthermore, it takes less polymer loading to reach a positive surface charge for pABOLs with higher molar mass. This is because polycations with higher molar mass have more effective binding sites per chain, leading to the increase in binding constant between the polymer chains and saRNA²⁷ and consequently more polymer incorporated into nanoparticles and increased surface charge. This indicates that higher molar mass is favourable for reaching the desired surface charge with a lower polymer loading, which could also be beneficial for endosomal escape, as more tertiary amines per nanoparticles can serve as a proton sponge.²⁸ Transmission electron microscopy (TEM) was used to confirm the nanoparticle structure. The particle size (pABOL-100/saRNA=45:1, w/w) shown in FIG. 2d is smaller than the hydrodynamic diameter demonstrated in FIG. 2b, which is commonly observed and caused by dehydration during sample characterization.

In order to evaluate the effect of pABOL molar mass on the intracellular delivery of saRNA, the inventors used saRNA encoding firefly luciferase (fLuc) as a reporter protein and indicator of transfection efficiency (FIG. 3a). PEI is a polycation that widely used for nucleic acid transfection, and thus serves as a positive control.²⁹ At weight ratios below 10:1, there was no effect of increasing the molar mass, likely because of surface charge being negative or neutral, as shown in FIG. 1c, which is unfavourable for cell uptake or colloidal stability. However, at weight ratios >45:1, the transfection efficiency shows a molar mass dependence—higher molar masses enhance higher transfection efficacy. To investigate further, additional in vitro tests were conducted using pABOLs with a wider range of molar masses at weight ratio of 45:1 (FIG. 3b). The results confirm the molar mass dependence, but also suggest non-monotonic behavior. At molar masses below 72 kg mol⁻¹, higher molar mass is indeed preferable. However, the transfection efficiency reaches a plateau for pABOLs with molar mass between 72 and 167 kg mol⁻¹. Considering the added time it takes to increase the molar mass from 100 to 167 kg mol⁻¹ during polymerization and negligible impact on transfection efficacy, there is no advantage in increasing the molar mass beyond 100 kg mol⁻¹.

The inventors also tested whether increasing the molar mass of pABOL similarly enhanced the transfection efficiency mRNA and plasmid DNA (pDNA). Although the enhancement in mRNA and pDNA transfection was not as significant as in saRNA, it implies the molar mass effect is not only specifically applied to long-chain nucleic acids, like saRNA, but to other nucleic acid species as well. This knowledge is useful for the future design of polymer-based delivery systems for nucleic acids.

In addition to transfection efficiency, the inventors also evaluated the in vitro cytotoxicity of saRNA/pABOL formulations (FIG. 3c). Compared to PEI, pABOLs display less cytotoxicity. Furthermore, a molar mass dependence was observed for pABOLs. pABOLs with low/moderate molar masses (8 and 25 kg mol⁻¹) demonstrate much less cytotoxicity, comparing to their high molar mass analogues (72 and 100 kg mol⁻¹), which could be due to surface charge and/or the concentration of free polycations.

The inventors then sought to determine the role of bioreduction of pABOLs on in vitro transfection efficiency. As a bioreducible polycation, it is hypothesized that pABOL releases saRNA via the intracellular glutathione (GSH) reduction of the disulfide bonds on its backbone.¹⁶ To confirm that pABOL is capable of being reduced by GSH, the bioreduction of pABOLs was monitored using GSH and the reduced product was identified to be a dithiol compound. The inventors then used a known GSH inhibitor, buthionine sulphoximine (BSO), 3° to pretreat cells and evaluate whether pABOLs had the same transfection with normal or reduced intracellular levels of GSH. All pABOL polyplexes showed a significant decrease in transfection efficiency following BSO pretreatment (FIG. 2d). PEI, which is not bioreducible, showed no decrease in transfection efficiency indicating that BSO pretreatment did not impact the ability of the cells to express luciferase, and that GSH is integral in decomplexation of pABOL.

This agrees with previous reports that the bioreducibility of pABOL accelerates the decomplexation with other nucleic acids.^{18,31} This may be particularly relevant for self-amplifying RNAs, where the fast release mechanism delivered by pABOLs may facilitate rapid transgene expression.

### Example 3—Optimization of pABOL/saRNA Complexation Procedure for Sterile Filtration and Scale-Up

In order to facilitate downstream product sterilization, it is imperative to develop saRNA-polyplexes that can undergo filter sterilization (0.2 μm) without loss of activity.

To address this issue, the inventors optimized a titration method to prepare polyplexes with a size of <100 nm. Titrating saRNA solutions (800 μL, 1.00×10⁻³ mg mL⁻¹) into polymer solutions (200 μL, 0.18 mg mL⁻¹) at a flow rate of 160 μL min⁻¹ yields smaller nanoparticles with a hydrodynamic diameter of ˜70 nm, narrow dispersity (0.2) and high surface charge (+23 mV) (FIG. 4a). Increasing the weight ratio from 45:1 to 60:1 did not influence particle sizes. The inventors found that a hydrodynamic diameter of ˜70 nm is sufficiently small enough for sterile filtration without losing any nanoparticles. The saRNA recovery after sterile filtration was monitored using Nanodrop at 260 nm (FIG. 4b). RNA concentrations before (black) and after (red) filtration were identical as was the hydrodynamic diameter (FIG. 4c), suggesting that no saRNA loss during sterile filtration

In order to demonstrate that polyplexes formed by the titration method enable high transfection efficiency even after sterile filtration, the inventors evaluated sterile filtered particles in vitro and in vivo. Polyplexes prepared using direct mixing were used as the control. While the transfection efficiency of polyplexes formed by direct mixing decreased by at least one order of magnitude (FIG. 4d) after sterile filtration, the titrated polyplexes were not affected at all (FIG. 4e), suggesting that nanoparticles of ˜70 nm and narrow dispersity are favorable for sterile filtration. A similar phenomenon was also observed in vivo; polyplexes formed via titration had high luciferase expression (˜10⁶ p/s) both before and after sterile filtration, while the ones generated by direct mixing had slightly lower luciferase expression (˜10⁵ p/s) and were no longer effective after sterile filtration (FIGS. 4f and 4g). Thus the inventors concluded that the titration method enables reproducible and scalable production of pABOL/saRNA polyplexes that facilitate protein expression both in vitro and in vivo.

### Example 4—Increasing pABOL Molar Mass Enhances Luciferase Expression In Vivo

The inventors further investigated whether increasing the molar mass of pABOLs enhanced the delivery and expression of saRNA in vivo, using fLuc as a reporter protein (FIG. 5). They tested a range of pABOL molecular weights, from 8 to 167 kg mol⁻¹, as these were the polymers that they found to effectively complex and condense saRNA (FIGS. 2b and 2c). Mice were injected with 5 μg of fLuc saRNA/pABOL polyplexes prepared at ratio of 45:1 (w/w) either intramuscularly (IM) or intradermally (ID) and imaged after 7 d, which has been previously shown to be peak protein expression for Venezuelan Equine encephalitis virus (VEEV).¹³ The inventors used two commercially available linear PEIs as a positive control—PEI MAX, which was used in all of their transfection experiments, and in vivo jetPEI®, which has previously been show to more effectively deliver RNA in vivo,³² The inventors observed signal from only one leg of one mouse in the PEI MAX IM group, whereas the average of the jetPEI group was 8×10⁴ p/s (FIG. 5a), confirming that jetPEI is a more effective in vivo delivery agent for RNA. The inventors observed similar superiority of pABOL polyplexes in vivo as for the in vitro experiments; the 8, 41, 72, 100 and 167 kg mol⁻¹ pABOLs had average luciferase expression of 5×10⁶ p/s when injected IM, ˜62-fold higher than the jetPEI group. Interestingly, the 25 kg mol⁻¹ pABOL had equivalent luciferase expression (˜10⁵ p/s) to jetPEI when injected IM, resulting in parabolic relationship between pABOL molar mass and luciferase expression in vivo. The 8, 100 and 167 kg mol⁻¹ groups had statistically significantly higher luciferase expression than the jetPEI when injected IM, with p=0.0446, 0.0332 and 0.0354, respectively. There was no signal from the ID jetPEI group (FIG. 5b). However, the pABOL ID mice had similarly high luciferase expression to the IM groups and the inventors again observed a parabolic relationship between expression and molar mass. The 8, 25 and 100 kg mol⁻¹ groups had luciferase expression of ˜10⁶ p/s, while the 41 and 72 kg mol⁻¹ groups had luciferase expression of ˜10⁵ p/s. However, only the 25 and 100 kg mol⁻¹ groups were statistically significantly higher, with p=0.0093 and 0.0186, respectively. Without wishing to be bound by theory, the inventors postulate that this parabolic relationship is governed by a mechanism wherein high luciferase expression from the 8 kg mol⁻¹ pABOL polyplexes results from more rapid reduction and thus rapid uptake of RNA in vivo, whereas the higher molar mass polymers are reduced less quickly but provide more adequate protection for the RNA potentially resulting high intracellular RNA delivery. Thus, the pABOLs with moderate molar mass (25 and 41 kg mol⁻¹), which theoretically are reduced less quickly but provide less adequate RNA protection result in lower signal and more variability of protein expression in vivo.

Finally, the inventors sought to determine the optimal ratio of pABOL to saRNA in vivo (FIG. 5e,f). They used ratios of 1:1 to 60:1 with pABOL-100, and observed that ratios of <25:1 yielded luciferase expression of 104 p/s or less. However, with a ratio of 45:1 the luciferase expression increased to ˜10⁶ p/s and there was no added benefit of increasing the ratio to 60:1. The inventors are the first to observe that increasing the molar mass of a cationic, bioreducible polymer enhances protein expression from saRNA in vivo.

### Example 5—pABOL Enhances the Quantity of Cells Expressing saRNA Both In Vivo and Ex Vivo in Human Skin Explants

After observing efficient saRNA delivery in vivo, the inventors then sought to investigate whether pABOLs enhance the quality or quantity of cells expressing saRNA both ex vivo in a clinically relevant human skin explant model and in vivo in mouse muscle and skin. For the skin explants, the inventors compared saRNA alone, the commercially available PEIs (PEI MAX and jetPEI) and 25, 72 and 100 kg mol⁻¹ pABOL complexed with 2 μg of enhanced green fluorescent protein (eGFP) saRNA (FIG. 6a,d). RNA alone resulted in eGFP expression in ˜1% of human skin cells (FIG. 6a), and complexation with PEI MAX and jetPEI did not increase the number of eGFP-positive cells. However, the 25 kg mol⁻¹ pABOL complexed with saRNA at a ratio of 10:1, 25:1 and 45:1 (w/w) increased the number of eGFP-positive cells to 3% and was statistically significantly higher than the RNA alone (p=0.0080, 0.0056 and 0.0457, respectively). Interestingly, the 72 kg mol⁻¹ pABOL did not increase the number of eGFP-positive cells at any of the ratios tested, but the 100 kg mol⁻¹ pABOL resulted in 1.5% and 2.5% eGFP-positive cells at ratios of 25:1 and 45:1 (p=0.0346 and 0.0170, respectively).

The inventors then sought to characterize whether the formulations were enhancing the amount of protein expression per cell as evidenced by quantifying the median fluorescence intensity (MFI) (FIG. 6d). RNA alone had an eGFP MFI of ˜10², and none of the formulations enhanced the protein expression per cell, which would manifest as a shift to the right on the x-axis of the histogram plot in FIG. 6d. It is hypothesized that this is due to the self-replicating nature of the VEEV vector, wherein upon entering a cell it exhausts the cellular translational machinery resulting in the maximum MFI per cell.

The inventors then tested whether increasing the molar mass of pABOL enhanced the number of cells expressing eGFP after IM and ID injection in mice (FIG. 6b,c). The inventors observed that RNA alone yielded expression in ˜10% of cells when injected IM, which was only enhanced to ˜20% of cells by 41 and 100 kg mol⁻¹ pABOL (p=0.0074 and 0.0022, respectively). 8 kg mol⁻¹ pABOL enhanced the eGFP+ cells to 16%, but this was not statistically significant. Similarly, the inventors observed that RNA alone yielded eGFP expression in 20% of cells after ID injection, which was increased to ˜30% of cells with 8 and 100 kg mol⁻¹ pABOL, although they were not statistically significant. jetPEI and 41 kg mol⁻¹ pABOL did not enhance the number of eGFP+ cells when injected ID. The inventors postulate that this is due to differential cell types between the muscle and the skin, which may have different kinetics of pABOL reduction and saRNA expression. Similar to the human skin explants, there was no significant shift in GFP MFI (FIG. 6e,f), further indicating that the total protein expression relies on the number of cells and not the amount of protein being expressed by each cell. These results directly reflect the relationship between pABOL molar mass and luciferase expression in vivo demonstrated in FIG. 5.

While the 25 kg mol⁻¹ pABOL resulted in ˜4% of eGFP-positive cells in human skin explants, this is a relatively low transfection efficiency. While this strongly agrees with the in vivo RNA expression levels that Liang et al. observed after intramuscular and intradermal mRNA injection in rhesus macaques,³³ the inventors sought to determine whether this correlated with luciferase expression in human skin explants. The inventors injected human skin explants with 2 μg of fLuc saRNA complexed with jetPEI, 25, 72 or 100 kg mol⁻¹ pABOL at a mass ratio of 45:1 (w/w). Indeed the 25 and 100 kg mol⁻¹ pABOL polyplexes had the highest luciferase expression (100,000 p/s), which directly reflect the percentage of eGFP+ cells in FIG. 5a. Overall, the inventors found that pABOL enhances the percentage of cells expressing saRNA compared to RNA alone or commercially available PEIs, when injected IM or ID in vivo in mice or ID ex vivo in human skin explants.

### Example 6—pABOL-Delivered saRNA is Preferentially Expressed by Epithelial Cells in Human Skin Explants

The inventors then further investigated which cells in human skin explants were expressing eGFP saRNA after intradermal injection (FIG. 7). The inventors observed that human skin explants are composed primarily of epithelial cells (53.7%), dendritic cells (14.8%), fibroblasts (11.6%) and Langerhans cells (10.8%) (FIG. 7a). The remaining 9% is composed of more rare immune cells, including leukocytes (4.0%), natural killer (NK) cells (2.6%), T cells (1.7%), B cells (0.6%) and monocytes (0.2%). Despite the predominance of epithelial cells in the skin when injected alone, saRNA was expressed in dendritic cells (DCs) (18.5%), leukocytes (16.3%), fibroblasts (16.0%), epithelial cells (13.1%), B cells (12.7%), Langerhans cells (12.3%), monocytes (8.0%), T cells (1.6%) and NK cells (1.4%) (FIG. 7b). There was a similar trend in cell types for both PEI MAX and jetPEI formulations. However, for all of the pABOL polyplexes, epithelial cells were the dominant cell type expressing the saRNA (18˜24%), followed by DCs, leukocytes, Langerhans cells, B cells, fibroblasts, monocytes, NK cells and T cells. The inventors hypothesize that the predominant uptake of RNA alone and PEIs by mostly immune cells indicates that these formulations are scavenged by professional immune cells, whereas the pABOL formulations may actively enhance cellular uptake into epithelial cells. These findings are similar to characterization by Liang et al. of which cells express mRNA encapsulated in lipid nanoparticle formulations when injected intradermally in rhesus macaques.³³ In that study they did not characterize the same cell types but found that the formulations were mostly taken up by and expressed in monocytes and DCs. It is unknown whether the total number or the phenotype of cells that express saRNA affect the immunogenicity of a vaccine. However, here the inventors show that formulating saRNA with pABOL results in uptake by a more diverse array of human skin cells compared to RNA alone or commercially available PEIs.

### Example 7—Hemagluttinin (HA) saRNA/pABOL Polyplexes Induce High HA Antibody Titers and Confer Complete Protection Against Flu Challenge In Vivo

The inventors then sought to assess the immunogenicity and protective capacity of HA-encoding saRNA delivered by pABOL, when injected either IM or ID (FIG. 8). Mice receive a prime and boost of either 1 or 0.1 μg of saRNA complexed with either jetPEI, 8 kg mol⁻¹ pABOL or 100 kg mol⁻¹ pABOL at a ratio of 45:1 (w/w). The boost was administered 6 weeks after the initial prime. The mice were challenged IN with Cal/09 flu virus three weeks after the boost and weighed daily to monitor disease progression.

The naïve mice in both the IM and ID groups all lost >25% of their body weight between days 4-6 and had to be culled according to the challenge protocol (FIG. 8a,b). In the IM injection groups, all mice in the PEI and 8 kg mol⁻¹ pABOL groups were completely protected, even in the 0.1 μg groups, with the 1 μg 8 kg mol⁻¹ pABOL group showing the least amount of weight loss at peak viremia (˜8%). All the mice in the 1 μg 100 kg mol⁻¹ pABOL group were completely protected, but two mice in the 0.1 μg 100 kg mol⁻¹ pABOL group reached 25% weight loss on day 5 and had to be culled, thus resulting in 60% survival in this group. The HA IgG antibody titers (FIG. 8b) directly reflect the challenge results; all groups show increasing antibody titers between 3 and 6 weeks, and then after the boost. The 1 μg 8 kDa pABOL group had the highest antibody titers (˜40,000 ng/mL) after 9 weeks, whereas the PEI and 100 kg mol⁻¹ pABOL groups that received 1 μg were equivalent (˜10,000 ng/mL). Even after 9 weeks, the 0.1 μg 100 kg mol⁻¹ pABOL group only reached a titer of ˜100 ng/mL, whereas the 8 kg mol⁻¹ pABOL and PEI groups reached titers of ˜8,000 and ˜2,000 ng/mL, respectively.

Compared to the IM injections, the ID injection groups were less protective against influenza challenge. Only the 1 μg PEI group conferred complete protection, and resulted in ˜12% weight loss during peak viremia. The 1 μg 8 kg mol⁻¹ pABOL had ˜20% weight loss after 5 days and only reached antibody titers of ˜500 ng/mL. The 0.1 μg PEI, 0.1 μg 8 kg mol⁻¹ pABOL and 1/0.1 μg 100 kg mol⁻¹ pABOL groups all had approximately equivalent antibody titers, never reaching more than −100 ng/mL and exhibiting low survival.

Overall, the 8 kg mol⁻¹ pABOL group exhibited the highest antibody levels against HA after IM injection and conferred complete protection against flu challenge, even at a dose of only 0.1 μg. These results show that route of administration (IM vs. ID) greatly influences the immunogenicity of polyplex-based vaccines, and that protein expression levels are not necessarily predictive of immunogenicity.

### Materials

All solvents and reagents were obtained from commercial sources (Aldrich and Fisher) and used as received unless stated otherwise. Dialysis tubing (14 kg mol⁻¹ molecular weight cut-off) was obtained from BioDesign Inc. of New York. Syringe filters with hydrophilic PVDF membrane were purchased from Sigma Aldrich.

### Methods

### Characterization

SEC: The molecular weights and dispersities were characterized using an Agilent PL GPC-50 instrument, equipped with a refractive index (RI) detector, with HPLC grade DMF (containing 0.075 wt % LiBr) as the eluent at a flow rate of 1.0 mL min⁻¹ at 40° C. Two GRAM Linear columns were used in series. Near monodispersed poly(methyl methacrylate) standards were used to calibrate the instrument. The poly(amino amide)s were dissolved in HPLC grade DMF, containing 0.075 wt % LiBr, and filtered through 0.2 μm syringe filters prior to analysis. Crude polymers were used for SEC characterization unless stated otherwise.

NMR: ¹H, ¹³C{¹H}, ¹H—¹H COSY and HSQC NMR spectra were recorded using a Bruker AV 400 MHz spectrometer at room temperature.

DLS: Dynamic light scattering was used to determine the hydrodynamic diameter (Dh) and polydispersity of the nanostructures formed between PABOLs and saRNA, in buffer solutions (20 Mm HEPES, 5 wt % glucose in water, pH 7.4), and was measured using the Zetasizer Nano ZS instrument. The scattering angle was fixed at 173°. Data processing was carried out using cumulant analysis of the experimental correlation function and the Stokes-Einstein equation was used to calculate the hydrodynamic radii. All solutions were analyzed using disposable polystyrene cuvettes.

Zeta potential: Zeta potential measurements were also conducted at 25° C. using a ZETASIZER Nano ZS instrument.

Nanodrop: The saRNA recovery was monitored using a Nanodrop One (Thermo Fisher) before and after sterile filtration of the polyplexes, through a 0.2 m syringe filter (membrane material: hydrophilic PVDF).

Mass spectroscopy: Mass spec characterizations were conducted using a Waters LCT Premier Mass Spectrometer. Samples were ionized using the electrospray (ES) technique.

TEM: Transmission electron microscopy (TEM) was performed on polyplexes that were prepared in H₂O. 10 μL of sample was pipetted directly onto a holey carbon film grid with 300 mesh copper (Agar Scientific, UK) and stained with 2% uranyl acetate, washed twice with DI H₂O and allowed to air dry. Samples were then imaged on a TEM-2100 Plus Electron Microscope (JEOL USA, Peabody, MA, USA) using a voltage of 80 kV.

### Improved Synthesis Procedure of PABOLs.

PABOL was synthesized by aza-Michael polyaddition of 4-amino-1-butanol (ABOL) to N,N′-cystaminebisacrylamide (CBA). In a typical experiment, CBA (221.0 mg, 0.848 mmol), ABOL (78 μL, 0.840 mmol) and trimethylamine (12 μL, 0.084 mmol) were added into an ampoule flask charged with a stir bar. A mixed solvent, MeOH/water (176 μL, 4/1, v/v), was also added into the ampoule flask. Polymerization was carried out in the dark at 45° C. under static nitrogen atmosphere. The reaction mixture became clear in less than 2 h. The mixture was allowed to react for 5 to 14 days (depending on the targeted molecular weight) to yield a highly viscous solution. Aliquots were taken at predetermined time intervals for ¹H NMR and SEC to monitor the conversion and molar mass. The reaction was stopped by MeOH dilution (50 mL) once the targeted molar mass was reached. The diluted reaction mixture was then acidified with 1.0 M HCl to pH ˜4, and then purified by dialysis against acidic water (4.0 L, pH ˜5, refreshed 6 times in 3 days). The polymers in their HCl-salt form were collected as white solid after freeze-dry.

### Synthesis of Fluorescence Labelled PABOL-100.

PABOL-100 (30 mg; 2.857×10⁻⁴ mmol) was dissolved in DMF (400 μL) in a vial charged with a stir bar, and 10 μL of TEA was added to promote the dissolution of PABOL chains. Then, NIR 797 isothiocyanate (1 mg, 1.140×10⁻³ mmol) was added into the polymer solution. The molar ratio of [—OH]/[isothiocyanate]=1/3.9. The mixture was allowed to react at 25° C. in dark for 18 h. The reaction mixture was then dialyzed in dark against dialysis against acidic water (500 mL, pH ˜5, refreshed 6 times in 3 days). The labelled PABOL-100 were collected as dark green solid after freeze-dry. The graft density was calculated to be 9.75% (1 label per 10.26 —OH) based on ¹H NMR spectrum.

### Protocols of PABOL Reduction in the Presence of GSH.

PABOL and GSH were dissolved in D₂O in a vial charged with a stir bar. The molar ratio of [S—S]/[GSH]=1/10. The mixture was allowed to react at 37° C. for 24 h. Aliquots were taken at predetermined time intervals for ¹H NMR and Mass spectroscopy to determine the conversion. After the complete reduction of the disulfide bond, HPLC technique was employed to separate GSH, GSSG and the degradation products, using mixed solvent of MeCN (with 0.1% TFA) and H₂O (with 0.1% TFA) at a flow rate of 10 mL/min using a Shimadzu HPLC instrument. The MeCN content of the mixed solvent increased gradiently from 5% to 30%.

In Vitro Transcription of saRNA.

Self-amplifying RNA derived from the Venezuelan Equine encephalitis Virus (VEEV) encoding firefly luciferase (fLuc), enhanced green fluorescent protein (eGFP) or hemagglutinin (HA) from the H1N1 A/California/07/2009 strain was produced using in vitro transcription. pDNA was transformed into E. coli and cultured in 50 mL of LB with 100 μg/mL carbenicillin (Sigma Aldrich, UK) and isolated using a Plasmid Plux MaxiPrep kit (QIAGEN, UK). pDNA concentration and purity was measured on a NanoDrop One (ThermoFisher, UK) and subsequently linearized using MluI for 2 h at 37° C. and heat inactivated at 80° C. for 20 min. For in vitro transfections, capped RNA was synthesized using 1 μg of linearized DNA template in a mMessage mMachine™ reaction (Promega, UK) and purified using a MEGAClear™ column (Promega, UK) according to the manufacturer's protocol. For in vivo experiments, uncapped in vitro RNA transcripts were synthesized using 1 μg of linearized DNA template in a MEGAScript™ reaction (Promega, UK) according to the manufacturer's protocol. Transcripts were then purified by overnight LiCl precipitation at −20° C., pelleted by centrifugation at 14,000 rpm for 20 min, washed 1× with 70% EtOH, centrifuged at 14,000 rpm for 5 min, and then resuspended in UltraPure H₂O. Purified transcripts were then capped using the ScriptCap™ m7G Capping System (CellScript, Madison, WI, USA) and ScriptCapt™ 2′-O-Methyltransferase Kit (CellScript, Madison, WI, USA) simultaneously according to the manufacturer's protocol. Capped transcripts were then purified by LiCl precipitation as detailed above, resuspended in UltraPure H₂O and stored at −80° C. until further use.

Polyplex Formation Between PABOL and saRNA.

Stock solutions of PEI, PABOLs and saRNA were prepared first by directly dissolving these materials in molecular grade water and stored in fridge. The concentration of the stock solutions are 2.00 μg/L (PEI), 0.24 μg/L (fLuc Mut RepRNA) and 5.00 μg/μL (PABOLs, in vitro studies) or 50 μg/μL (PABOLs, in vivo studies), respectively. Polyplexes were prepared using two methods: a) ‘direct mixing’ and b) ‘titration’.

a) Direct mixing: In a typical procedure, 4.17 μL of the saRNA stock solution was diluted to 200 μL using the HEPES buffer (20 mM HEPES, 5 wt % glucose in water, pH 7.4). A predetermined amount of polymer stock solution was also diluted to 800 μL using the same buffer. Each tube was vortexed and centrifuged to ensure the homogeneity. Then, the polymer buffer solution was added to the saRNA buffer solution rapidly, following with vortex for 20 s to form the complex. A series of complex solutions were prepared with the polymer/saRNA weight ratio ranging from 1/1 to 60/1.

b) Titration: In a typical procedure, 4.17 μL of the saRNA stock solution was diluted to 800 μL using the HEPES buffer (20 mM HEPES, 5 wt % glucose in water, pH 7.4). A predetermined amount of polymer stock solution was also diluted to 200 μL using the same buffer in centrifuge tubes, equipped with stir bars. Each tube was placed on a stir plate and stirred at 1200 rpm at ambient temperature. Then, the RNA solution was added to the polymer solution at a rate of 160 μL/min (unless otherwise stated). A series of complex solutions were prepared with the polymer/saRNA weight ratios ranging from 1/1 to 60/1.

### Protocols for In Vitro Transfection Studies.

Transfections were performed in HEK293T.17 cells (ATCC, USA) that were maintained in culture in complete Dulbecco's Modified Eagle's Medium (cDMEM) (Gibco, Thermo Fisher, UK) containing 10% fetal calf serum (FCS), 5 mg/mL L-glutamine and 5 mg/mL penicillin/streptomycin (Thermo Fisher, UK). Cells were plated at a density of 50,000 cells per well in a clear 96 well plate 24 h prior to transfection. For the transfection, the media was completely removed and replace with 50 μL of pre-warmed transfection medium (DMEM with 5 mg/mL L-glutamine). 100 μL of the polyplex solution was added to each well and allowed to incubate for four hours, then the transfection media was completely removed and replaced with 100 μL of cDMEM. After 24 h from the initial transfection, 50 μL of media was removed from each well and 50 μL of ONE-Glo™ D-luciferin substrate (Promega, UK) was added and mixed well by pipetting. The total volume was transferred to a white 96-well plate (Costar) and analyzed on a FLUOstar Omega plate reader (BMG LABTECH, UK) and background from the media control wells was subtracted. For the glutathione inhibition assay, cells were incubated with 200 μM buthionine sulfoximine (BSO), a known glutathione inhibitor,³⁴ for 4 hours prior to the transfection; and then the transfection was performed as detailed above.

### Cytotoxicity of Polyplexes.

For analysis of polyplex cytotoxicity, cells were transfected with varying ratios of PABOL and PEI to saRNA ranging from 10:1 to 450:1 (w/w) according to the above protocol. 24 h after the initial transfection, 20 μL of CellTiter-Blue reagent (Promega, UK) was added to each well and allowed to incubate for 1 h. The plate was then analyzed for absorbance on a FLUOstar Omega plate reader (BMG LABTECH, UK) and normalized to the media control.

In Vivo fLuciferase Expression in Mice.

All animals were handled in accordance with the UK Home Office Animals Scientific Procedures Act 1986 and with an internal ethics board and UK government approved project and personal license. Food and water were supplied ad libitum. Female BALB/c mice (Charles River, UK) 6-8 weeks of age were placed into groups (n=5) and housed in a fully acclimatized room. Mice were injected intramuscularly (IM) in both hind legs or intradermally (ID) with 5 μg of fLuc saRNA in a total volume of 50 μL. After 7 days, the mice were injected intraperitoneally (IP) with 100 μL of XenoLight RediJect D-Luciferin Substrate (Perkin Elmer, UK) and allowed to rest for 10 min. Mice were then anesthetized using isoflurane and imaged on an In Vivo Imaging System (IVIS) FX Pro (Kodak Co., Rochester, NY, USA) equipped with Molecular Imaging Software Version 5.0 (Carestream Health, USA) for 2 min. Signal from each injection site was quantified using Molecular Imaging software and expressed as relative light units (p/s).

Flow Cytometry Analysis of eGFP Expression in Human Skin Explants.

Surgically resected specimens of human skin tissue were collected at Charing Cross Hospital, Imperial NHS Trust, London, UK. All tissues were collected after receiving signed informed consent from patients, under protocols approved by the Local Research Ethics Committee. The tissue was obtained from patients undergoing elective abdominoplasty or mastectomy surgeries. Tissue was refrigerated until arrival in the laboratory where the subcutaneous layer of fat was removed, and the tissue was excised into 1 cm² sections. Explants were incubated at 37° C. with 5% CO₂ in petri dishes with 10 mL of cDMEM. Media was replaced daily. Explants were injected intradermally (ID) using a Micro-Fine Demi 0.3 mL syringe (Becton Dickinson, UK) with 2 μg of eGFP saRNA/pABOL polyplexes in a volume of 100 μL. After three days, skin explants were minced well with scissors and incubated in 3 mL DMEM supplemented with 1 mg/mL collagenase P (Sigma, UK) and 5 mg/mL dispase II (Sigma, UK) for 4 h at 37° C. on a rotational shaker. Digests were then filtered through a 70 μm cell strainer and centrifuged at 1750 RPM for 5 min. Cells were then resuspended in 1 mL of FACS buffer (PBS+2.5.% FCS) at a concentration of 1E7 cells/mL. 100 μL of cell suspension was added to a FACS tube and stained with Fixable Aqua Live/Dead Cell stain (Thermo Fisher, UK) dilution 1:400 in FACS buffer for 20 min on ice. Cells were then washed with 2.5 mL of FACS buffer, centrifuged at 1750 rpm for 5 min and stained with a panel of antibody to identify each cell type, as described in Supplementary Table 1, for 30 min. Cells were then washed with 1 mL of FACS buffer, centrifuged at 1750 rpm for 5 min and resuspended in 250 μL PBS. Cells were fixed by addition of 250 μL of 3.0% paraformaldehyde for a final concentration of 1.5%, and refrigerated until flow cytometry analysis. Samples were analyzed on a LSRFortessa™ (BD Biosciences, UK) with FACSDiva software (BD Biosciences, UK) with 100,000 acquired live cell events. Gating was performed as previously described.³⁵ Phenotypic identity of GFP-positive cells was quantified using FlowJo Version 10 (FlowJo LLC, Oregon, USA).

Flow Cytometry Analysis of eGFP Expression in Murine Skin and Muscle.

Female BALB/c mice (Charles River, UK) 6-8 weeks of age were placed into groups (n=5) and housed in a fully acclimatized room. Mice were injected intramuscularly (IM) in both hind legs or intradermally (ID) with 5 μg of eGFP saRNA in a total volume of 50 μL. After 7 days, the mice were culled and the muscle or skin around the injection site was excised and put in 3 mL DMEM supplemented with 1 mg/mL collagenase P (Sigma, UK) and 5 mg/mL dispase II (Sigma, UK) for 4 h at 37° C. on a rotational shaker. Digests were then filtered through a 70 μm cell strainer and centrifuged at 1750 RPM for 5 min. Cells were then resuspended in 1 mL of FACS buffer (PBS+2.5.% FCS) at a concentration of 1E7 cells/mL. 100 μL of cell suspension was added to a FACS tube and stained with Fixable Aqua Live/Dead Cell stain (Thermo Fisher, UK) dilution 1:400 in FACS buffer for 20 min on ice. Cells were then washed with 1 mL of FACS buffer, centrifuged at 1750 rpm for 5 min and resuspended in 250 μL PBS. Cells were fixed by addition of 250 μL of 3.0% paraformaldehyde for a final concentration of 1.5%, and refrigerated until flow cytometry analysis. Samples were analyzed on a LSRFortessa™ (BD Biosciences, UK) with FACSDiva software (BD Biosciences, UK) with 100,000 acquired live cell events. Phenotypic identity of GFP-positive cells was quantified using FlowJo Version 10 (FlowJo LLC, Oregon, USA).

Ex Vivo fLuciferase Expression in Human Skin Explants.

Human skin tissue was collected and excised as described above. Explants were incubated at 37° C. with 5% CO₂ in petri dishes with 10 mL of cDMEM. Media was replaced daily. Explants were injected intradermally (ID) using a Micro-Fine Demi 0.3 mL syringe (Becton Dickinson, UK) with 2 μg of fLuc saRNA/pABOL polyplexes in a volume of 100 μL. After three days, skin explants were inverted and the media was replaced with 5 mL of cDMEM supplemented with 100 μL of XenoLight RediJect D-Luciferin Substrate (Perkin Elmer, UK) and imaged on an In Vivo Imaging System (IVIS) FX Pro (Kodak Co., Rochester, NY, USA) equipped with Molecular Imaging Software Version 5.0 (Carestream Health, USA) for 60 min. Signal from each injection site was quantified using Molecular Imaging software and expressed as relative light units (p/s).

In Vivo Immunogenicity of HA saRNA.

BALB/c mice were immunized IM in one hind leg with either 1 or 0.1 μg of HA saRNA formulated with either in vivo jet-PEI®, PABOL-8 (Table 1, #2) or PABOL-100 (Table 1, #8) in a total volume of 50 μL, and boosted after 6 weeks. Blood was collected after 3, 6 and 9 weeks from study onset via tail bleeding, centrifuged at 10,000 rpm for 5 min and then the serum was removed and stored at −80° C. until further use.

### HA-Specific ELISA.

A semi-quantitative immunoglobulin ELISA protocol was performed as previously described.³⁶ Briefly, 0.5 μg/mL of HA coated ELISA plates were blocked with 1% BSA/0.05% Tween-20 in PBS. After washing, diluted samples were added to the plates and incubated for 2 h, washed, and a 1:4,000 dilution of anti-mouse IgG-HRP (Southern Biotech, UK) was used. Standards were prepared by coating ELISA plate wells with anti-mouse Kappa (1:1,000) and Lambda (1:1,000) light chain (Serotec, UK), blocking with PBS/1% BSA/0.05% Tween-20, washing and adding purified IgG (Southern Biotech, UK) starting at 1,000 ng/mL and titrating down with a 5-fold dilution series. Samples and standard were developed using TMB (3,3′-5,5′-tetramethylbenzidine) and the reaction was stopped after 5 min with Stop solution (Insight Biotechnologies, UK). Absorbance was read on a spectrophotometer (VersaMax, Molecular Devices) with SoftMax Pro GxP v5 software.

### Influenza Challenge.

3 weeks after the boost injection, mice were challenge with XXX pfu of influenza (Cal/09) suspending in 100 uL of PBS. Mice were anesthetized using isoflurane, challenged intranasally (IN), and weighed each day to determine weight loss. According to challenge protocol, mice were culled if they sustained more than three days of 20% weight loss or one day of 75% weight loss.

### Statistical Analysis.

Graphs and statistics were prepared in GraphPad Prism, version 8. Statistical differences were analyzed using either a two-tailed t test or an ordinary one-way ANOVA with multiple comparisons, with α=0.05 used to indicate significance.

### REFERENCES

1 Ulmer, J. B., Valley, U. & Rappuoli, R. Vaccine manufacturing: challenges and solutions. Nat. Biotechnol. 24, 1377-1383, doi:10.1038/nbt1261 (2006).
2 Petsch, B. et al. Protective efficacy of in vitro synthesized, specific mRNA vaccines against influenza A virus infection. Nature Biotechnology 30, 1210, doi:10.1038/nbt.2436 https://www.nature.com/articles/nbt.2436 #supplementary-information (2012).
3 Guan, S. & Rosenecker, J. Nanotechnologies in delivery of mRNA therapeutics using nonviral vector-based delivery systems. Gene Therapy 24, 133, doi:10.1038/gt.2017.5 (2017).
4 Karikó, K. et al. Incorporation of Pseudouridine Into mRNA Yields Superior Nonimmunogenic Vector With Increased Translational Capacity and Biological Stability. Mol. Ther. 16, 1833-1840, doi:10.1038/mt.2008.200 (2008).
5 Thess, A. et al. Sequence-engineered mRNA Without Chemical Nucleoside Modifications Enables an Effective Protein Therapy in Large Animals. Mol. Ther. 23, 1456-1464, doi:10.1038/mt.2015.103 (2015).
6 Kauffman, K. J., Webber, M. J. & Anderson, D. G. Materials for non-viral intracellular delivery of messenger RNA therapeutics. Journal of Controlled Release 240, 227-234, doi:https://doi.org/10.1016/j.jconrel.2015.12.032 (2016).
7 Sahin, U., Karikó, K. & Tureci, Ö. mRNA-based therapeutics—developing a new class of drugs. Nat. Rev. Drug. Discov. 13, 759-780, doi:10.1038/nrd4278 (2014).
8 Perri, S. et al. An Alphavirus Replicon Particle Chimera Derived from Venezuelan Equine encephalitis and Sindbis Viruses Is a Potent Gene-Based Vaccine Delivery Vector. Journal of Virology 77, 10394-10403, doi:10.1128/JVI.77.19.10394-10403.2003 (2003).
9 Chahal, J. S. et al. Dendrimer-RNA nanoparticles generate protective immunity against lethal Ebola, HiN1 influenza, and Toxoplasma gondii challenges with a single dose. Proceedings of the National Academy of Sciences of the United States of America 113, E4133-E4142, doi:10.1073/pnas.1600299113 (2016).
10 Geall, A. J., Verma, A. & Otten, G. R. Nonviral delivery of self-amplifying RNA vaccines. Proceedings of the National Academy of Sciences, doi:10.1073/pnas.1209367109 (2012).
11 Bogers, W. M. et al. Potent immune responses in rhesus macaques induced by non-viral delivery of a self-amplifying RNA vaccine expressing HIV-1 envelope with a cationic nanoemulsion. Journal ofInfectious Diseases, doi:10.1093/infdis/jiu522 (2014).
12 Brito, L. A. et al. A Cationic Nanoemulsion for the Delivery of Next-generation RNA Vaccines. Mol. Ther. 22, 2118-2129, doi:10.1038/mt.2014.133 (2014).
13 Vogel, A. B. et al. Self-Amplifying RNA Vaccines Give Equivalent Protection against Influenza to mRNA Vaccines but at Much Lower Doses. Mol. Ther. 26, 446-455, doi:https://doi.org/10.1016/j,ymthe.2017.11.017 (2018).
14 Blakney, A. K., Yilmaz, G., McKay, P. F., Becer, C. R. & Shattock, R. J. One Size Does Not Fit All: The Effect of Chain Length and Charge Density of Poly(ethylene imine) Based Copolymers on Delivery of pDNA, mRNA, and RepRNA Polyplexes. Biomacromolecules 19, 2870-2879, doi:10.1021/acs.biomac.8b00429 (2018).
15 Kunath, K. et al. Low-molecular-weight polyethylenimine as a non-viral vector for DNA delivery: comparison of physicochemical properties, transfection efficiency and in vivo distribution with high-molecular-weight polyethylenimine. Journal of Controlled Release 89, 113-125, doi:https://doi.org/10.1016/S0168-3659(03)00076-2 (2003).
16 Lin, C. et al. Novel Bioreducible Poly(amido amine)s for Highly Efficient Gene Delivery. Bioconjugate Chemistry 18, 138-145, doi:10.1021/bc0602001 (2007).
17 Chen, G. et al. Combining Fluorination and Bioreducibility for Improved siRNA Polyplex Delivery. ACS Appl. Mater. Interfaces 9, 4457-4466, doi:10.1021/acsami.6b14184 (2017).
18 Chen, G. et al. Fluorination Enhances Serum Stability of Bioreducible Poly(amido amine) Polyplexes and Enables Efficient Intravenous siRNA Delivery. Adv. Healthc. Mater. 7, 1700978, doi:10.1002/adhm.201700978 (2018).
19 Piest, M. et al. Novel poly(amido amine)s with bioreducible disulfide linkages in their diamino-units: Structure effects and in vitro gene transfer properties. J. Control. Release 130, 38-45, doi:10.1016/j.jconrel.2008.05.023 (2008).
Burr, M. L. et al. CMTM6 maintains the expression of PD-L¹ and regulates anti-tumour immunity. Nature 549, 101-105, doi:10.1038/nature23643 https://www.nature.cornarticles/nature23643 #supplementary-information (2017).
21 Houtkooper, R. H. et al. Mitonuclear protein imbalance as a conserved longevity mechanism. Nature 497, 451-457, doi:10.1038/nature12188 https://www.nature.com/articles/nature12188 #supplementary-information (2013).
22 Stampfel, G. et al. Transcriptional regulators form diverse groups with context-dependent regulatory functions. Nature 528, 147-151, doi:10.1038/nature15545 https://www.nature.com/aricles/nature15545 #supplementary-information (2015).
23 Delafosse, L., Xu, P. & Durocher, Y. Comparative study of polyethylenimines for transient gene expression in mammalian HEK293 and CHO cells. Journal of Biotechnology 227, 103-111, doi:https://dol.org/10.1016/j.biotec.2016.04.028 (2016).
24 Mann, J. F. S. et al. Pulmonary delivery of DNA vaccine constructs using deacylated PEI elicits immune responses and protects against viral challenge infection. J. Control. Release 170, 452-459, doi:https://doi.org/10.1016/j.jconrel2013.06.004 (2013).
25 Lin, C. et al. Bioreducible poly(amido amine)s with oligoamine side chains: Synthesis, characterization, and structural effects on gene delivery. J. Control. Release 126, 166-174, doi:10.1016/j.jconrel.2007.11.012 (2008).
26 Wang, S. W. et al. Biomimetic Fluorocarbon Surfactant Polymers Reduce Platelet Adhesion on PTFE/ePTFE Surfaces. Journal of Biomaterials Science—Polymer Edition 20, 619-635, doi:10.1163/156856209x426439 (2009).
27 Lounis, F. M. et al. Interactions between Oppositely Charged Polyelectrolytes by Isothermal Titration Calorimetry: Effect of Ionic Strength and Charge Density. J. Phys. Chem. B 121, 2684-2694, doi:10.1021/acs.jpcb.6b11907 (2017).
28 Varkouhi, A. K., Scholte, M., Storm, G. & Haisma, H. J. Endosomal escape pathways for delivery of biologicals. J. Control. Release 151, 220-228, doi:https://doi.org/10.1016/j.jconrel.2010.11.004 (2011).
29 Miyata, K., Nishiyama, N. & Kataoka, K. Rational design of smart supramolecular assemblies for gene delivery: chemical challenges in the creation of artificial viruses. Chem. Soc. Rev. 41, 2562-2574, doi:10.1039/c1cs15258k (2012).
30 Tagde, A., Singh, H., Kang, M. H. & Reynolds, C. P. The glutathione synthesis inhibitor buthionine sulfoximine synergistically enhanced melphalan activity against preclinical models of multiple myeloma. Blood Cancer J. 4, e229-e229, doi:10.1038/bcj.2014.45 (2014).
31 Bauhuber, S., Hozsa, C., Breunig, M. & Gopferich, A. Delivery of Nucleic Acids via Disulfide-Based Carrier Systems. Adv. Mater. 21, 3286-3306, doi:10.1002/adma.200802453 (2009).
32 Kaczmarek, J. C. et al. Polymer-Lipid Nanoparticles for Systemic Delivery of mRNA to the Lungs. Angewandte Chemie (International ed. in English) 55, 13808-13812, doi:10.1002/anie.201608450 (2016).
33 Liang, F. et al. Efficient Targeting and Activation of Antigen-Presenting Cells In Vivo after Modified mRNA Vaccine Administration in Rhesus Macaques. Mol. Ther. 25, 2635-2647, doi:https://doi.org/10.1016/j.ymthe.2017.08.006 (2017).
34 Tagde, A., Singh, H., Kang, M. H. & Reynolds, C. P. The glutathione synthesis inhibitor buthionine sulfoximine synergistically enhanced melphalan activity against preclinical models of multiple myeloma. Blood Cancer Journal 4, e229, doi:10.1038/bcj.2014.45 https://www.nature.com/articles/bcj20144F supplementary-information (2014).
35 Blakney, A. K. et al. The Skin You Are In: Design-of-Experiments Optimization of Lipid Nanoparticle Self-Amplifying RNA Formulations in Human Skin Explants. ACS Nano 13, 5920-5930, doi:10.1021/acsnan0.9b01774 (2019).
36 Badamchi-Zadeh, A. et al. Intramuscular Immunisation with Chlamydial Proteins Induces Chlamydia trachomatis Specific Ocular Antibodies. PLoS ONE 10, e0141209, doi:10.1371/journal.pone.0141209 (2015).

1. A polymeric composition comprising a plurality of polymers of formula (I): 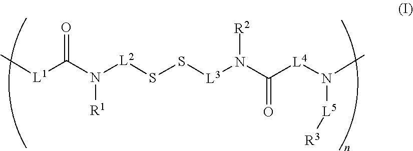
wherein L¹ to L⁵ are each independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms;
L⁶ and L⁷ are independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene or an optionally substituted 5 to 10 membered heteroarylene, wherein adjacent carbon atoms in the alkylene, alkenylene or alkynylene are optionally interrupted by one or more heteroatoms;
R¹ and R² are each independently H, an optionally substituted C₁₋₁₂ alkyl, an optionally substituted C₂₋₁₂ alkenyl or an optionally substituted C₂₋₁₂ alkynyl;
R³ is —OR⁴, —COOR⁴, —SO₂OR⁴, (OCH₂CH₂)ₘOH, or NR⁴R⁵,
R⁴ and R⁵ are each independently H, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl, wherein adjacent carbon atoms in the alkyl, alkenyl or alkenyl are optionally interrupted by one or more heteroatoms; and
m is an integer between 1 and 10;
or a pharmaceutically acceptable complex, salt, solvate, tautomeric form or polymorphic form thereof;
characterised in that the average molecular mass of the plurality of polymers of formula (I) is greater than 5 kg mol⁻¹.

2. The polymeric composition according to claim 1, wherein L¹ to L⁴ are each independently an optionally substituted C₁₋₆ alkylene, an optionally substituted C₂₋₆ alkenylene or an optionally substituted C₂₋₆ alkynylene.

3-14. (canceled)

15. A polymeric composition comprising self-amplifying RNA (saRNA) and a plurality of polymers of formula (I): 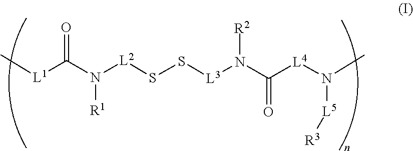
wherein L¹ to L⁵ are each independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms;
L⁶ and L⁷ are independently an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene or an optionally substituted 5 to 10 membered heteroarylene, wherein adjacent carbon atoms in the alkylene, alkenylene or alkynylene are optionally interrupted by one or more heteroatoms;
R¹ and R² are each independently H, an optionally substituted C₁₋₁₂ alkyl, an optionally substituted C₂₋₁₂ alkenyl or an optionally substituted C₂₋₁₂ alkynyl;
R³ is —OR⁴, —COOR⁴, —SO₂OR⁴, (OCH₂CH₂)ₘOH, or NR⁴R⁵,
R⁴ and R⁵ are each independently H, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl, wherein adjacent carbon atoms in the alkyl, alkenyl or alkenyl are optionally interrupted by one or more heteroatoms; and
m is an integer between 1 and 10;
or a pharmaceutically acceptable complex, salt, solvate, tautomeric form or polymorphic form thereof.

16-27. (canceled)

28. A method of producing a high molar mass poly(amido amine), the method comprising contacting a compound of formula (II): 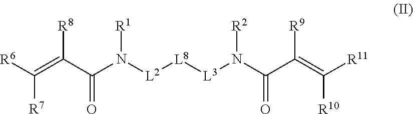
with a compound of formula (III):
NHR¹²R¹³ (III)
in the presence of a Lewis base to thereby cause the compounds of formula (II) and formula (III) to undergo a polymerisation reaction and produce the high molar mass poly(amido amine);
wherein R¹ and R² are each independently H, an optionally substituted C₁₋₁₂ alkyl, an optionally substituted C₂₋₁₂ alkenyl or an optionally substituted C₂₋₁₂ alkynyl;
R⁶ to R¹¹ are each independently H, a halogen, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl or an optionally substituted 5 to 10 membered heteroaryl;
L² and L³ are each independently absent or an optionally substituted C₁₋₁₂ alkylene, an optionally substituted C₂₋₁₂ alkenylene, an optionally substituted C₂₋₁₂ alkynylene, an optionally substituted C₃₋₆ cycloalkylene, an optionally substituted 3 to 8 membered heterocyclylene, an optionally substituted C₆₋₁₂ arylene, an optionally substituted 5 to 10 membered heteroarylene or L⁶L⁷, wherein adjacent carbon atoms in the alkylene, alkenylene or alkynylene are optionally interrupted by one or more heteroatoms;
L⁸ is absent or is —S—S—;
R¹² is an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl, an optionally substituted 5 to 10 membered heteroaryl, wherein adjacent carbon atoms in the alkyl, alkenyl or alkenyl are optionally interrupted by one or more heteroatoms; and
R¹³ is H, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, an optionally substituted 3 to 8 membered heterocyclyl, an optionally substituted C₆₋₁₂ aryl, an optionally substituted 5 to 10 membered heteroaryl, wherein adjacent carbon atoms in the alkyl, alkenyl or alkenyl are optionally interrupted by one or more heteroatoms.

29. The method of claim 28, wherein the compound of formula (II) is a compound of formula (IIa):

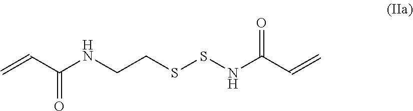

30-32. (canceled)
